SEQUENCE LISTING (10 sequences)

SEQ ID NO: 1 - protein, 15 aa, Artificial Sequence
  Pembrolizumab, VL-CDR1
RASKGVSTSGYSYLH

SEQ ID NO: 2 - protein, 7 aa, Artificial Sequence
  Pembrolizumab, VL-CDR2
LASYLES

SEQ ID NO: 3 - protein, 9 aa, Artificial Sequence
  Pembrolizumab, VL-CDR3
QHSRDLPLT

SEQ ID NO: 4 - protein, 111 aa, Artificial Sequence [sequence omitted]

SEQ ID NO: 5 - protein, 218 aa, Artificial Sequence
  Pembrolizumab, light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, Artificial Sequence
  Pembrolizumab, VH-CDR1
NYYMY

SEQ ID NO: 7 - protein, 17 aa, Artificial Sequence
  Pembrolizumab, VH-CDR2
GINPSNGGTNFNEKFKN

SEQ ID NO: 8 - protein, 11 aa, Artificial Sequence
  Pembrolizumab, VH-CDR3
RDYRFDMGFDY

SEQ ID NO: 9 - protein, 120 aa, Artificial Sequence [sequence omitted]

SEQ ID NO: 10 - protein, 447 aa, Artificial Sequence
  Pembrolizumab, heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2023/0250182 A1
(43) Pub. Date: Aug. 10, 2023

(54) METHODS FOR TREATING CANCER OR VON-HIPPEL LINDAU DISEASE USING A COMBINATION OF A PD-1 ANTAGONIST, A HIF-2 ALPHA INHIBITOR, AND LENVATINIB OR A PHARMACEUTICALLY ACCEPTABLE SALT THEREOF

(71) Applicants: Merck Sharp & Dohme LLC, Rahway, NJ (US); Eisai R&D Management Co., Ltd., Tokyo (JP)

(72) Inventors: Rodolfo Fleury Perini, Wynnewood, PA (US); Elaine M. Pinheiro, Needham, MA (US); Jacqueline Willemann-Rogerio, West Orange, NJ (US)

(73) Assignees: Merck Sharp & Dohme LLC, Rahway, NJ (US); Eisai R&D Management Co., Ltd., Tokyo (JP)

(21) Appl. No.: 18/009,109

(22) PCT Filed: Jun. 21, 2021
(86) PCT No.: PCT/US21/38171

§ 371 Date: Dec. 8, 2022

Related U.S. Application Data
(60) Provisional application No. 63/146,926, filed on Feb. 8, 2021.
(60) Provisional application No. 63/042,225, filed on Jun. 22, 2020.

(51) Int. Cl. C07K 16/28; A61K 31/47; A61K 31/277; A61P 35/00

(52) U.S. Cl. CPC C07K 16/2896; A61K 31/47; A61K 31/277; A61P 35/00; A61K 2039/545

(57) ABSTRACT

Provided herein are methods of treating cancer (e.g., RCC) or von-Hippel Lindau disease, which comprise administering to a human patient in need thereof: (a) a PD-1 antagonist; (b) a HIF-2α inhibitor; and (c) lenvatinib, or a pharmaceutically acceptable salt thereof. Also provided are kits containing such agents and uses of therapeutic combinations of such agents for the treatment of cancer.

### FIELD OF INVENTION

Provided herein are methods for treating cancer (e.g., renal cell carcinoma (RCC)) or von-Hippel Lindau disease, using a combination of (a) a programmed death 1 protein (PD-1) antagonist, (b) a hypoxia-inducible factor 2a (HIF-2α) inhibitor, and (c) lenvatinib, or a pharmaceutically acceptable salt thereof.

### REFERENCE TO SEQUENCE LISTING SUBMITTED ELECTRONICALLY

The sequence listing of the present application is submitted electronically via EFS-Web as an ASCII formatted sequence listing with a file name “25062WOPCT-SEQLIST-09APR2021_ST25.txt”, creation date of Apr. 9, 2021, and a size of 10 KB. This sequence listing submitted via EFS-Web is part of the specification and is herein incorporated by reference in its entirety.

### BACKGROUND OF THE INVENTION

PD-1 is recognized as an important player in immune regulation and the maintenance of peripheral tolerance. Immune checkpoint therapies targeting PD-1 or its ligand (e.g., PD-L1) have resulted in groundbreaking improvements in clinical response in multiple human cancer types (Brahmer et al., N Engl J Med, 366: 2455-2465 (2012); Garon et al., N Engl J Med, 372:2018-2028 (2015); Hamid et al., N Engl J Med, 369:134-144 (2013); Robert et al., Lancet, 384:1109-1117 (2014); Robert et al., N Engl J Med, 372: 2521-2532 (2015); Robert et al., N Engl J Med, 372:320-330 (2015); Topalian et al., N Engl J Med, 366:2443-2454 (2012); Topalian et al., J Clin Oncol, 32:1020-1030 (2014); Wolchok et al., N Engl J Med, 369:122-133 (2013)). Immune therapies targeting the PD-1 axis include monoclonal antibodies directed to the PD-1 receptor (e.g., KEYTRUDA® (pembrolizumab), Merck and Co., Inc., Kenilworth, N.J.; OPDIVO® (nivolumab), Bristol-Myers Squibb Company, Princeton, N.J.); LIBTAYO® (cemiplimab), Regeneron Pharmaceuticals, Inc., Tarrytown, N.Y.; TYVYT® (sintilmab), Innovent Biologics, Inc., Jiangsu, China; tislelizumab, BeiGene, Beijing, China; camrelizumab, Hengrui Therapeutics, Inc., Princeton, N.J.; and toripalimab, Junshi Biosciences, Shanghai, China); and those that bind to the PD-L1 ligand (e.g., IMFINZI® (durvalumab), AstraZeneca Pharmaceuticals LP, Wilmington, Del.; and BAVENCIO® (avelumab), Pfizer Inc, New York, N.Y.).

Intratumoral hypoxia is a driving force in cancer progression and is closely linked to poor patient prognosis and resistance to chemotherapy and radiation treatment. Hypoxia-Inducible Factors (HIF-1a and HIF-2α) are transcription factors that play central roles in the hypoxic response pathway. Under normoxic conditions, the tumor suppressor von Hippel-Lindau (VHL) protein binds to specific hydroxylated proline residues and recruits the E3 ubiquition-ligase complex that targets HIF-α proteins for proteasomal degradation. Under hypoxic conditions, HIF-α proteins accumulate and enter the nucleus to stimulate the expression of genes that regulate anaerobic metabolism, angiogenesis, cell proliferation, cell survival, extracellular matrix remodeling, pH homeostasis, amino acid and nucleotide metabolism, and genomic instability. VHL deficiency can also result in accumulated HIF expression under oxygenated conditions (pseudohypoxic conditions). Accordingly, directly targeting HIF-α proteins offers an exciting opportunity to attack tumors on multiple fronts (Keith, et al., Nature Rev. Cancer 12: 9-22, 2012).

Specifically, HIF-2a is a key oncogenic driver in clear cell renal cell carcinoma (ccRCC) (Kondo, K., et al., Cancer Cell, 1:237-246 (2002); Maranchie, J. et al, Cancer Cell, 1:247-255 (2002); Kondo, K., et al., PLoS Biol., 1:439-444 (2003)). In mouse ccRCC tumor models, knockdown of HIF-2α expression in pVHL (von Hippel-Lindau protein) defective cell lines blocked tumor growth comparable to reintroduction of pVHL. In addition, expression of a stabilized variant of HIF-2α was able to overcome the tumor suppressive role of pVHL. Belzutifan, or a pharmaceutically acceptable salt thereof, a novel HIF-2α inhibitor with excellent in vitro potentcy, pharmacokinetic profile and in vivo efficacy in mouse models, has shown encouraging outcomes in patients with advanced renal cell carcinoma (Xu, Rui, et al., J. Med. Chem. 62:6876-6893 (2019).

HIF-2α has emerged as a key HIF isoform that is essential for von Hippel Lindau (VHL) deficient ccRCC. In VHL deficient ccRCC xenograft mouse tumor models, knockdown of HIF-2α expression inhibits tumor formation comparable with reintroduction of functional VHL and overexpression of HIF2α alone can rescue the tumor-suppressive effect of VHL (see, Kondo K., Klco J, Nakamura E, Lechpammer M, Kaelin W G Jr. Inhibition of HIF is necessary for tumor suppression by the von Hippel-Lindau protein. Cancer Cell 2002; 1:237-46; Maranchie J K, Vasselli J R, Riss J, Bonifacino J S, Linehan W M, Klausner R D. The contribution of VHL substrate binding and HIF1-alpha to the phenotype of VHL loss in renal cell carcinoma. Cancer Cell 2002; 1:247-55; Kondo K, Kim W Y, Lechpammer M, Kaelin W G Jr. Inhibition of HIF2alpha is sufficient to suppress pVHL-defective tumor growth. PLoS Biol 2003; 1:E83; Zimmer M, Doucette D, Siddiqui N, Iliopoulos O. Inhibition of hypoxia-inducible factor is sufficient for growth suppression of VHL−/− tumors. Mol Cancer Res 2004; 2:89-95). These data suggest that HIF-2α may be the tumorigenic driver in ccRCC. HIF proteins can also be activated in many other types of cancers (ex. breast, liver, colon, brain, pancreatic cancers) due to the tumor hypoxic microenvironment and have been implicated in cancer initiation, progression and metastasis (see, Jarman E J, Ward C, Turnbull A K, Martinez-Perez C, Meehan J, Xintaropoulou C, Sims A H, Langdon S P. HER2 regulates HIF-2α and drives an increased hypoxic response in breast cancer. Breast Cancer Res. 2019 Jan. 22; 21(1):10; Wigerup C, Pahlman S., Bexell D. Therapeutic targeting of hypoxia and hypoxia-inducible factors in cancer. Pharmacology & Therapeutics 164 (2016) 152-169). HIF-2α has been shown to be stabilized by host tumor cells under tumor hypoxia, including endothelial cells, perivascular tumor cells, and immune suppressive cell types such as tumor associated macrophages (TAMs) where it plays a role in regulating innate immunity (see, Imtiyaz H Z, Williams E P, Hickey M M, Patel S A, Durham A C, Yuan L J, Hammond R, Gimotty P A, Keith B, Simon M C. Hypoxia-inducible factor 2alpha regulates macrophage function in mouse models of acute and tumor inflammation. J Clin Invest. 2010 August; 120(8):2699-714). However, little is known about which VHL proficient tumor types and combination strategies make theoretical sense to explore with an inhibitor of HIF-2α.

Von-Hippel Lindau disease (VHL disease) is an autosomal dominant syndrome that not only predisposes patients to kidney cancer (˜70% lifetime risk), but also to hemangioblastomas, pheochromocytoma and pancreatic neuroendocrine tumors. VHL disease results in tumors with constitutively active HIF-α proteins with the majority of these dependent on HIF-2α activity (Maher, et al. Eur. J. Hum. Genet. 19: 617-623, 2011). HIF-2α has been linked to cancers of the retina, adrenal gland and pancreas through both VHL disease and activating mutations. Recently, gain-of-function HIF-2α mutations have been identified in erythrocytosis and paraganglioma with polycythemia (Zhuang, et al. NEJM 367: 922-930, 2012; Percy, et al. NEJM 358: 162-168, 2008; and Percy, et al. Am. J. Hematol. 87: 439-442, 2012). Notably, a number of known HIF-2α-target gene products (e.g., VEGF, PDGF, and cyclin D1) have been shown to play pivotal roles in cancers derived from kidney, liver, colon, lung, and brain. In fact, therapies targeted against one of the key HIF-2α regulated gene products, VEGF, have been approved for the treatment of these cancers.

Tyrosine kinases are involved in the modulation of growth factor signaling and thus are important targets for cancer therapies. Lenvatinib is a multiple RTK (multi-RTK) inhibitor that selectively inhibits the kinase activities of vascular endothelial growth factor (VEGF) receptors (VEGFR1 (FLT1), VEGFR2 (KDR) and VEGFR3 (FLT4)), and fibroblast growth factor (FGF) receptors FGFR1, 2, 3 and 4 in addition to other proangiogenic and oncogenic pathway-related RTKs (including the platelet-derived growth factor (PDGF) receptor PDGFRα; KIT; and the RET proto-oncogene (RET)) involved in tumor proliferation. In particular, lenvatinib possesses a new binding mode (Type V) to VEGFR2, as confirmed through X-ray crystal structural analysis, and exhibits rapid and potent inhibition of kinase activity, according to kinetic analysis.

It has been proposed that the efficacy of anti-PD-1 or anti-PD-L1 antagonistic antibodies might be enhanced if administered in combination with other approved or experimental cancer therapies, e.g., radiation, surgery, chemotherapeutic agents, targeted therapies, agents that inhibit other signaling pathways that are disregulated in tumors, and other immune enhancing agents. However, there are no clear guidelines as to which agent combined with the anti-PD-1 or anti-PD-L1 antibodies may be effective or in which patients the combination may enhance the efficacy of treatment. Thus, there is an unmet need in the art for high efficacy therapeutic combinations that can generate a robust immune response to cancer.

### SUMMARY

The present disclosure provides methods of treating cancer (e.g., RCC) or von-Hippel Lindau disease, using a combination of a PD-1 antagonist, a HIF-2α inhibitor, and lenvatinib, or a pharmaceutically acceptable salt thereof.

The present disclosure further provides kits including a PD-1 antagonist, a HIF-2α inhibitor, and lenvatinib, or a pharmaceutically acceptable salt thereof.

Also provided herein are uses of a therapeutic combination for treating cancer (e.g., RCC) or von-Hippel Lindau disease, and the therapeutic combination includes a PD-1 antagonist, a HIF-2α inhibitor, and lenvatinib, or a pharmaceutically acceptable salt thereof.

In one aspect, provided herein is a method of treating cancer or von-Hippel Lindau disease, comprising administering to a human patient in need thereof:
(a) a PD-1 antagonist;
(b) a HIF-2α inhibitor; and
(c) lenvatinib, or a pharmaceutically acceptable salt thereof.

In some embodiments, the cancer is selected from the group consisting of bladder cancer, breast cancer, non-small cell lung cancer (NSCLC), colorectal cancer (CRC), renal cell carcinoma (RCC), hepatocellular carcinoma (HCC), pancreatic cancer and melanoma.

In certain embodiments, the cancer is metastatic. In some embodiments, the cancer is relapsed. In other embodiments, the cancer is refractory. In yet other embodiments, the cancer is relapsed and refractory.

In one embodiment, the cancer is bladder cancer. In another embodiment, the cancer is breast cancer. In yet another embodiment, the cancer is NSCLC. In still another embodiment, the cancer is CRC. In one embodiment, the cancer is RCC. In another embodiment, the cancer is HCC. In yet another embodiment, the cancer is pancreatic cancer. In yet another embodiment, the cancer is melanoma.

In one embodiment, the cancer is advanced RCC. In another embodiment, the cancer is metastatic RCC. In yet another embodiment, the cancer is relapsed RCC. In still another embodiment, the cancer is refractory RCC. In yet still another embodiment, the cancer is relapsed and refractory RCC.

In another aspect, provided herein is a kit comprising:
(a) a PD-1 antagonist;
(b) a HIF-2α inhibitor; and
(c) lenvatinib, or a pharmaceutically acceptable salt thereof.

In certain embodiments, the kit further comprises instructions for administering to a human patient the PD-1 antagonist, the HIF-2α inhibitor, and lenvatinib, or a pharmaceutically acceptable salt thereof.

In still another aspect, provided herein is use of a therapeutic combination for treating cancer in a human patient, wherein the therapeutic combination comprises:
(a) a PD-1 antagonist;
(b) a HIF-2α inhibitor; and
(c) lenvatinib, or a pharmaceutically acceptable salt thereof.

In some embodiments, the cancer is selected from the group consisting of bladder cancer, breast cancer, non-small cell lung cancer (NSCLC), colorectal cancer (CRC), renal cell carcinoma (RCC), hepatocellular carcinoma (HCC), pancreatic cancer and melanoma.

In certain embodiments, the cancer is metastatic. In some embodiments, the cancer is relapsed. In other embodiments, the cancer is refractory. In yet other embodiments, the cancer is relapsed and refractory.

In one embodiment, the cancer is bladder cancer. In another embodiment, the cancer is breast cancer. In yet another embodiment, the cancer is NSCLC. In still another embodiment, the cancer is CRC. In one embodiment, the cancer is RCC. In another embodiment, the cancer is HCC. In yet another embodiment, the cancer is pancreatic cancer. In yet another embodiment, the cancer is melanoma.

In one embodiment, the cancer is advanced RCC. In another embodiment, the RCC is advanced RCC with clear cell component (ccRCC). In yet another embodiment, the cancer is metastatic RCC. In yet another embodiment, the cancer is relapsed RCC. In still another embodiment, the cancer is refractory RCC. In yet still another embodiment, the cancer is relapsed and refractory RCC.

In one embodiment, the human patient has not received prior systemic treatment for advanced disease. In a class of the embodiment, the human patient has not received prior systemic treatment for advanced RCC.

In certain embodiments of various methods, kits, or uses provided herein, the PD-1 antagonist is an anti-human PD-1 monoclonal antibody or antigen binding fragment thereof.

In other embodiments of various methods, kits, or uses provided herein, the PD-1 antagonist is an anti-human PD-L1 monoclonal antibody or antigen binding fragment thereof.

In some embodiments of various methods, kits, or uses provided herein, the anti-human PD-1 monoclonal antibody is a humanized antibody.

In other embodiments of various methods, kits, or uses provided herein, the anti-human PD-1 monoclonal antibody is a human antibody.

In certain embodiments of various methods, kits, or uses provided herein, the HIF-2α inhibitor is belzutifan, or a pharmaceutically acceptable salt thereof.

In one embodiment of various methods, kits, or uses provided herein, the anti-human PD-1 monoclonal antibody is selected from the group consisting of pembrolizumab, nivolumab, cemiplimab, sintilimab, tislelizumab, camrelizumab and toripalimab.

In one embodiment of various methods, kits, or uses provided herein, the anti-human PD-1 monoclonal antibody is pembrolizumab.

In another embodiment of various methods, kits, or uses provided herein, the anti-human PD-1 monoclonal antibody is nivolumab.

In another embodiment of various methods, kits, or uses provided herein, the anti-human PD-1 monoclonal antibody is cemiplimab.

In another embodiment of various methods, kits, or uses provided herein, the anti-human PD-1 monoclonal antibody is sintilimab.

In another embodiment of various methods, kits, or uses provided herein, the anti-human PD-1 monoclonal antibody is tislelizumab.

In another embodiment of various methods, kits, or uses provided herein, the anti-human PD-1 monoclonal antibody is camrelizumab.

In another embodiment of various methods, kits, or uses provided herein, the anti-human PD-1 monoclonal antibody is toripalimab.

In another embodiment of various methods, kits, or uses provided herein, the anti-human PD-L1 monoclonal antibody is durvalumab.

In another embodiment of various methods, kits, or uses provided herein, the anti-human PD-L1 monoclonal antibody is avelumab.

In yet still another embodiment of various methods, kits, or uses provided herein, the lenvatinib or a pharmaceutically acceptable salt thereof is lenvatinib mesylate. Capsules for oral administration contain 4 mg or 10 mg of lenvatinib, equivalent to 4.90 mg or 12.25 mg of lenvatinib mesylate, respectively. In another embodiment, when a pharmaceutically acceptable salt of lenvatinib is administered, such as lenvatinib mesylate, and the dose of lenvatinib to be used is 4 mg, a medical practitioner would know to administer 4.90 mg of lenvatinib mesylate. In another embodiment, when a pharmaceutically acceptable salt of lenvatinib is administered, such as lenvatinib mesylate, and the dose of lenvatinib to be used is 10 mg, a medical practitioner would know to administer 12.25 mg of lenvatinib mesylate. In embodiments of various methods described herein, the human patient is administered 8, 10, 12, 14, 18, 20, or 24 mg lenvatinib once daily.

In one specific embodiment of various methods, kits, or uses provided herein, the PD-1 antagonist is pembrolizumab; and the HIF-2α inhibitor is belzutifan, or a pharmaceutically acceptable salt thereof.

In one specific embodiment of various methods, kits, or uses provided herein, the PD-1 antagonist is nivolumab; and the HIF-2α inhibitor is belzutifan, or a pharmaceutically acceptable salt thereof.

In one specific embodiment of various methods, kits, or uses provided herein, the PD-1 antagonist is cemiplimab; and the a HIF-2α inhibitor is belzutifan, or a pharmaceutically acceptable salt thereof.

In some embodiments of various methods described herein, the human patient is administered 200 mg, 240 mg, or 2 mg/kg pembrolizumab, and pembrolizumab is administered once every three weeks. In one embodiment, the human patient is administered 200 mg pembrolizumab once every three weeks. In one embodiment, the human patient is administered 240 mg pembrolizumab once every three weeks. In one embodiment, the human patient is administered 2 mg/kg pembrolizumab once every three weeks.

In certain embodiments of various methods described herein, the human patient is administered 400 mg pembrolizumab, and pembrolizumab is administered once every six weeks.

In other embodiments of various methods described herein, the human patient is administered 240 mg or 3 mg/kg nivolumab once every two weeks, or 480 mg nivolumab once every four weeks. In one specific embodiment, the human patient is administered 240 mg nivolumab once every two weeks. In one specific embodiment, the human patient is administered 3 mg/kg nivolumab once every two weeks. In one specific embodiment, the human patient is administered 480 mg nivolumab once every four weeks.

In other embodiments of various methods described herein, the human patient is administered 350 mg cemiplimab, and cemiplimab is administered once every three weeks.

In other embodiments of various methods described herein, the human patient is administered 800 mg of avelumab, and avelumab is administered once every two weeks.

In other embodiments of various methods described herein, the human patient is administered 10 mg/kg of durvalumab, and durvalumab is administered once every two weeks. In other embodiments of various methods described herein, the human patient is administered 1500 mg of durvalumab, and durvalumab is administered once every three weeks. In other embodiments of various methods described herein, the human patient is administered 1500 mg of durvalumab, and durvalumab is administered once every four weeks.

In still other embodiments of various methods described herein, the HIF-2α inhibitor is belzutifan, or a pharmaceutically acceptable salt thereof, and the human patient is administered from 40 mg to 120 mg of belzutifan, or a pharmaceutically acceptable salt thereof daily. In some embodiments, the human patient is administered 40, 80, or 120 mg of belzutifan, or a pharmaceutically acceptable salt thereof once daily. In one specific embodiment, the human patient is administered 40 mg of belzutifan, or a pharmaceutically acceptable salt thereof once daily. In another specific embodiment, the human patient is administered 80 mg of belzutifan, or a pharmaceutically acceptable salt thereof once daily. In another specific embodiment, the human patient is administered 120 mg of belzutifan, or a pharmaceutically acceptable salt thereof once daily.

Thus, in some embodiments, the human patient is administered:
(a) 200 mg, 240 mg, or 2 mg/kg pembrolizumab once every three weeks;
(b) 40, 80, or 120 mg of belzutifan once daily; and
(c) 8, 10, 12, 14, 18, 20, or 24 mg lenvatinib once daily.

In certain embodiments, the human patient is administered:
(a) 200 mg pembrolizumab once every three weeks;
(b) 120 mg of belzutifan once daily; and
(c) 20 mg lenvatinib once daily.

In certain embodiments, the human patient is administered:
(a) 240 mg pembrolizumab once every three weeks;
(b) 120 mg of belzutifan once daily; and
(c) 20 mg lenvatinib once daily.

In certain embodiments, the human patient is administered:
(a) 2 mg/kg pembrolizumab once every three weeks;
(b) 120 mg of belzutifan once daily; and
(c) 20 mg lenvatinib once daily.

In certain embodiments, the human patient is administered:
(a) 400 mg pembrolizumab once every six weeks;
(b) 120 mg of belzutifan once daily; and
(c) 20 mg lenvatinib once daily.

In a specific embodiment, provided herein is a method of treating RCC, comprising administering to a human patient in need thereof:
(a) 200 mg pembrolizumab once every three weeks;
(b) 120 mg of belzutifan once daily; and
(c) 20 mg lenvatinib once daily.

In certain embodiments of such a method, the anti-human PD-1 monoclonal antibody, HIF-2α inhibitor and lenvatinib are administered on the same day. In some embodiments, the anti-human PD-1 monoclonal antibody, the HIF-2α inhibitor, and lenvatinib are administered sequentially. In other embodiments, the anti-human PD-1 monoclonal antibody, the HIF-2α inhibitor, and lenvatinib are administered concurrently.

In some embodiments of various methods, kits, or uses described herein, the pharmaceutically acceptable salt of lenvatinib—lenvatinib mesylate—can be used. When lenvatinib mesylate is used, the dosage of lenvatinib mesylate is appropriately adjusted to provide equal mole of lenvatinib as 8, 10, 12, 14, 18, 20, or 24 mg lenvatinib provides.

### BRIEF DESCRIPTION OF THE DRAWINGS

FIG. 1 illustrates a scheme of the design overview the Phase 1b/2 Study of belzutifan, or a pharmaceutically acceptable salt thereof (MK-6482) in combination with pembrolizumab and lenvatinib in patients with 1^{st} line Advanced RCC (Experimental Arm A4).

FIG. 2A illustrates the anti-tumor effect of concurrent administration of a PD-1 antagonist, Lenvatinib and a HIF-2α inhibitor (MK-6482) as shown by average tumor volumes in each treatment group. Experimental details are described in Example 2.

FIG. 2B illustrates the anti-tumor effect of concurrent administration of a PD-1 antagonist, Lenvatinib and a HIF-2α inhibitor (MK-6482) as shown by average tumor volumes in the individual growth curves for each respective group. Experimental details are described in Example 2.

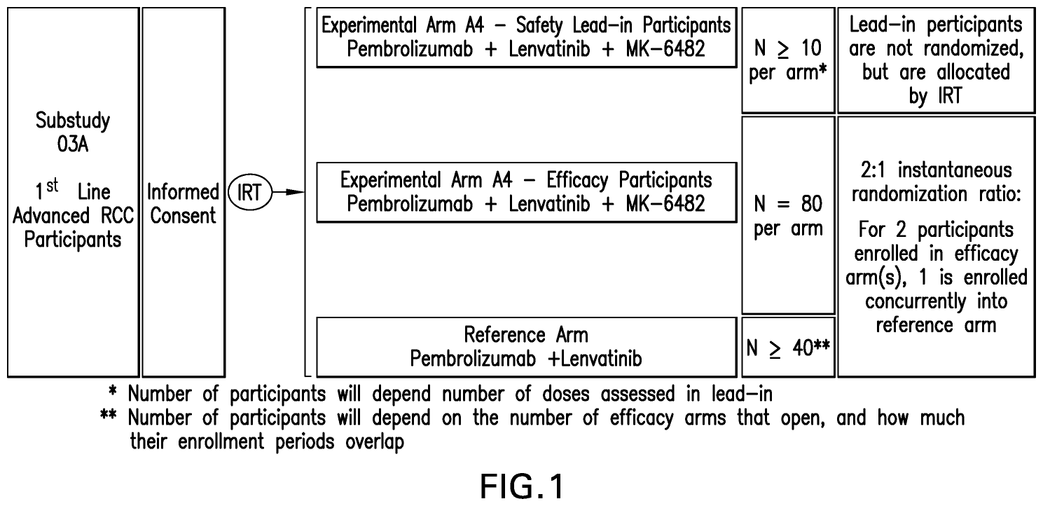

### DETAILED DESCRIPTION OF THE INVENTION

### 1. Definitions

Certain technical and scientific terms are specifically defined below. Unless specifically defined elsewhere in this document, all other technical and scientific terms used herein have the meaning commonly understood by one of ordinary skill in the art to which this disclosure relates.

“About” when used to modify a numerically defined parameter (e.g., the dose of an anti-PD-1 antibody or antigen binding fragment thereof, a HIF-2α inhibitor or antigen binding fragment thereof, or lenvatinib, or the length of treatment time with a combination therapy described herein) means that the parameter is within 20%, within 15%, within 10%, within 9%, within 8%, within 7%, within 6%, within 5%, within 4%, within 3%, within 2%, within 1%, or less of the stated numerical value or range for that parameter; where appropriate, the stated parameter may be rounded to the nearest whole number. For example, a dose of about 5 mg/kg may vary between 4.5 mg/kg and 5.5 mg/kg.

As used herein, including the appended claims, the singular forms of words such as “a,” “an,” and “the,” include their corresponding plural references unless the context clearly dictates otherwise.

The terms “administration” or “administer” refers to the act of injecting or otherwise physically delivering a substance as it exists outside the body (e.g., an anti-PD-1 antibody, HIF-2α inhibitor, and lenvatinib as described herein) into a patient, such as by oral, mucosal, intradermal, intravenous, subcutaneous, intramuscular delivery, and/or any other methods of physical delivery described herein or known in the art.

“PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, medicaments and disclosed uses in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively. The PD-1 antagonist is not anti-PD-L1 monoclonal antibody atezolizumab.

“HIF-2α inhibitor” means any chemical compound or biological molecule that inhibits the activity of HIF-2α. Alternative names or synonyms for HIF-2α include but are not limited to: hypoxia-inducible factor-2alpha, endothelial PAS domain-containing protein 1, and EPAS1.

As used herein, the term “antibody” refers to any form of immunoglobulin molecule that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic. As used herein, the term “antibody” encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

“Variable regions” or “V region” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “V_{H}.” The variable region of the light chain may be referred to as “V_{L}.” Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody V_{H} β-sheet framework, or one of three hypervariable regions (L1, L2, or L3) within the non-framework region of the antibody V_{L} β-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved b-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art and shown below in Table 1. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

TABLE 1
Correspondence between the CDR Numbering Systems

| | Kabat + | | | | | |
|---|---|---|---|---|---|---|
| | Chothia | IMGT | Kabat | AbM | Chothia | Contact |
| V_{H} CDR1 | 26-35 | 27-38 | 31-35 | 26-35 | 26-32 | 30-35 |
| V_{H} CDR2 | 50-65 | 56-65 | 50-65 | 50-58 | 52-56 | 47-58 |
| V_{H} CDR3 | 95-102 | 105-117 | 95-102 | 95-102 | 95-102 | 93-101 |
| V_{L} CDR1 | 24-34 | 27-38 | 24-34 | 24-34 | 24-34 | 30-36 |
| V_{L} CDR2 | 50-56 | 56-65 | 50-56 | 50-56 | 50-56 | 46-55 |
| V_{L} CDR3 | 89-97 | 105-117 | 89-97 | 89-97 | 89-97 | 89-96 |

“Chimeric antibody” refers to an antibody in which a portion of the heavy and/or light chain contains sequences derived from a particular species (e.g., human) or belonging to a particular antibody class or subclass, while the remainder of the chain(s) is derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences or derivatives thereof. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences or derivatives thereof, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” may be added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, unless otherwise indicated, “antibody fragment” or “antigen binding fragment” refers to a fragment of an antibody that retains the ability to bind specifically to the antigen, e.g., fragments that retain one or more CDR regions. An antibody that “specifically binds to” PD-1 or ILT4 is an antibody that exhibits preferential binding to PD-1 or ILT4 (as appropriate) as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, will bind to the target protein with an affinity that is at least two-fold greater, preferably at least ten times greater, more preferably at least 20-times greater, and most preferably at least 100-times greater than the affinity with non-target proteins.

Antigen binding portions include, for example, Fab, Fab′, F(ab′)2, Fd, Fv, fragments including CDRs, and single chain variable fragment antibodies (scFv), and polypeptides that contain at least a portion of an immunoglobulin that is sufficient to confer specific antigen binding to the antigen (e.g., PD-1 or ILT4). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

As used herein, the terms “at least one” item or “one or more” item each include a single item selected from the list as well as mixtures of two or more items selected from the list.

As used herein, the term “immune response” relates to any one or more of the following: specific immune response, non-specific immune response, both specific and non-specific response, innate response, primary immune response, adaptive immunity, secondary immune response, memory immune response, immune cell activation, immune cell-proliferation, immune cell differentiation, and cytokine expression.

The term “subject” (alternatively “patient”) as used herein refers to a mammal that has been the object of treatment, observation, or experiment. The mammal may be male or female. The mammal may be one or more selected from the group consisting of humans, bovine (e.g., cows), porcine (e.g., pigs), ovine (e.g., sheep), capra (e.g., goats), equine (e.g., horses), canine (e.g., domestic dogs), feline (e.g., house cats), lagomorphs (e.g., rabbits), rodents (e.g., rats or mice), Procyon lotor (e.g., raccoons). In particular embodiments, the subject is human.

The term “subject in need thereof” as used herein refers to a subject diagnosed with or suspected of having cancer or an infectious disease as defined herein.

The therapeutic agents and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The therapeutic agents and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific therapeutic agent being used, the rate of absorption desired, specific formulation or dosage form used, type or severity of the disorder being treated, the specific site of action, and conditions of the patient, and can be readily selected by a person skilled in the art.

The term “variant” when used in relation to an antibody (e.g., an anti-PD-1 antibody) or an amino acid region within the antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a variant of an anti-PD-1 antibody may result from one or more (such as, for example, about 1 to about 25, about 1 to about 20, about 1 to about 15, about 1 to about 10, or about 1 to about 5) changes to an amino acid sequence of a native or previously unmodified anti-PD-1 antibody. Variants may be naturally occurring or may be artificially constructed. Polypeptide variants may be prepared from the corresponding nucleic acid molecules encoding the variants. In specific embodiments, an antibody variant (e.g., an anti-PD-1 antibody variant) at least retains the antibody functional activity. In specific embodiments, an anti-PD-1 antibody variant binds to PD-1 and/or is antagonistic to PD-1 activity.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2 below.

TABLE 2
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared×100. For example, if 8 of 10 of the positions in two sequences are matched when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

“RECIST 1.1 Response Criteria” as used herein means the definitions set forth in Eisenhauer, E. A. et al., Eur. J. Cancer 45:228-247 (2009) for target lesions or nontarget lesions, as appropriate based on the context in which response is being measured.

“Sustained response” means a sustained therapeutic effect after cessation of treatment as described herein. In some embodiments, the sustained response has a duration that is at least the same as the treatment duration, or at least 1.5, 2.0, 2.5 or 3 times longer than the treatment duration.

“Treat” or “treating” cancer as used herein means to administer a therapeutic combination of an anti-human PD-1 monoclonal antibody or antigen binding fragment thereof, a HIF-2α inhibitor, and lenvatinib or a pharmaceutically acceptable salt thereof, to a subject having cancer or diagnosed with cancer to achieve at least one positive therapeutic effect, such as, for example, reduced number of cancer cells, reduced tumor size, reduced rate of cancer cell infiltration into peripheral organs, or reduced rate of tumor metastasis or tumor growth. Such “treatment” may result in a slowing, interrupting, arresting, controlling, or stopping of the progression of cancer as described herein but does not necessarily indicate a total elimination of the cancer or the symptoms of the cancer. Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by a combination therapy of the disclosure is any of PR, CR, OR, PFS, DFS, and OS. PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some embodiments, response to a combination therapy of the disclosure is any of PR, CR, PFS, DFS, or OR that is assessed using RECIST 1.1 response criteria. The treatment regimen for a combination therapy of the disclosure that is effective to treat a cancer patient may vary according to factors such as the disease state, age, and weight of the patient, and the ability of the therapy to elicit an anti-cancer response in the subject. While an embodiment of any of the aspects of the disclosure may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

As used herein, the terms “combination,” “combination therapy,” and “therapeutic combination” refer to treatments in which at least one anti-human PD-1 monoclonal antibody or antigen-binding fragment thereof, a HIF-2α inhibitor, and lenvatinib or a pharmaceutically acceptable salt thereof, and optionally additional therapeutic agents, each are administered to a patient in a coordinated manner, over an overlapping period of time. The period of treatment with the at least one anti-human PD-1 monoclonal antibody (or antigen-binding fragment thereof) (the “anti-PD-1 treatment”) is the period of time that a patient undergoes treatment with the anti-human PD-1 monoclonal antibody (or antigen-binding fragment thereof); that is, the period of time from the initial dosing with the anti-human PD-1 monoclonal antibody (or antigen-binding fragment thereof) through the final day of a treatment cycle. Similarly, the period of treatment with the HIF-2α inhibitor (the “HIF-2α inhibitor treatment”) is the period of time that a patient undergoes treatment with the HIF-2α inhibitor; that is, the period of time from the initial dosing with the HIF-2α inhibitor through the final day of a treatment cycle. The period of treatment with lenvatinib or a pharmaceutically acceptable salt thereof (the “lenvatinib treatment”) is the period of time that a patient undergoes treatment with lenvatinib; that is, the period of time from the initial dosing with lenvatinib through the final day of a treatment cycle. In the methods and therapeutic combinations described herein, the anti-PD-1 treatment overlaps by at least one day with the HIF-2α inhibitor treatment and overlaps by at least one day with the lenvatinib treatment. In certain embodiments, the anti-PD-1 treatment, the HIF-2α inhibitor treatment, and the lenvatinib treatment are the same period of time. In some embodiments, the anti-PD-1 treatment begins prior to the HIF-2α inhibitor and/or the lenvatinib treatment. In other embodiments, the anti-PD-1 treatment begins after the HIF-2α inhibitor and/or the lenvatinib treatment. In yet other embodiments, the HIF-2α inhibitor treatment begins prior to the anti-PD-1 and/or the lenvatinib treatment. In still other embodiments, the HIF-2α inhibitor treatment begins after the anti-PD-1 and/or the lenvatinib treatment. In some embodiments, the lenvatinib treatment begins prior to the HIF-2α inhibitor and/or the anti-PD-1 treatment. In other embodiments, the lenvatinib treatment begins after the HIF-2α inhibitor and/or the anti-PD-1 treatment. In certain embodiments, the anti-PD-1 treatment is terminated prior to termination of the HIF-2α inhibitor and/or the lenvatinib treatment. In other embodiments, the anti-PD-1 treatment is terminated after termination of the HIF-2α inhibitor and/or the lenvatinib treatment. In yet other embodiments, the HIF-2α inhibitor treatment is terminated prior to termination of the anti-PD-1 and/or the lenvatinib treatment. In still other embodiments, the HIF-2α inhibitor treatment is terminated after termination of the anti-PD-1 and/or the lenvatinib treatment. In certain embodiments, the lenvatinib treatment is terminated prior to termination of the HIF-2α inhibitor and/or the anti-PD-1 treatment. In other embodiments, the lenvatinib treatment is terminated after termination of the HIF-2α inhibitor and/or the anti-PD-1 treatment.

The terms “treatment regimen,” “dosing protocol,” and “dosing regimen” are used interchangeably to refer to the dose and timing of administration of each therapeutic agent in a combination therapy of the disclosure.

“Tumor” as it applies to a subject diagnosed with, or suspected of having, a cancer refers to a malignant or potentially malignant neoplasm or tissue mass of any size, and includes primary tumors and secondary neoplasms. Non-limiting examples of tumors include solid tumor (e.g., sarcoma (such as chondrosarcoma), carcinoma (such as colon carcinoma), blastoma (such as hepatoblastoma), etc.) and blood tumor (e.g., leukemia (such as acute myeloid leukemia (AML)), lymphoma (such as DLBCL), multiple myeloma (MM), etc.).

The term “tumor volume” or “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CT or MRI scans.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations of ±1%, ±2%, ±3%, ±4%, ±5%, ±10%, ±15%, and ±20% and their numerical equivalents. All ranges also are intended to include all included sub-ranges, although not necessarily explicitly set forth. For example, a range of 3 to 7 days is intended to include 3, 4, 5, 6, and 7 days. In addition, the term “or,” as used herein, denotes alternatives that may, where appropriate, be combined; that is, the term “or” includes each listed alternative separately as well as their combination.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the claims.

Unless otherwise defined, all technical and scientific terms used herein have the same meaning as commonly understood by one of ordinary skill in the art to which this disclosure relates. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present disclosure. The materials, methods, and examples are illustrative only and not intended to be limiting.

### 2. PD-1 Antagonist

Provided herein are PD-1 antagonists that can be used in the various methods, kits, and uses disclosed herein, including any chemical compound or biological molecule that blocks binding of PD-L1 to PD-1 and preferably also blocks binding of PD-L2 to PD-1.

Any monoclonal antibodies that bind to a PD-1 polypeptide, a PD-1 polypeptide fragment, a PD-1 peptide, or a PD-1 epitope and block the interaction between PD-1 and its ligand PD-L1 or PD-L2 can be used. In some embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1. In other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L2. In yet other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1 and the interaction between PD-1 and PD-L2.

Any monoclonal antibodies that bind to a PD-L1 polypeptide, a PD-L1 polypeptide fragment, a PD-L1 peptide, or a PD-L1 epitope and block the interaction between PD-L1 and PD-1 can also be used.

In certain embodiments, the anti-human PD-1 monoclonal antibody is selected from the group consisting of pembrolizumab, nivolumab, cemiplimab, sintilimab, tiselizumab, camrelizumab, toripalimab, pidilizumab (U.S. Pat. No. 7,332,582), AMP-514 (MedImmune LLC, Gaithersburg, Md.), PDR001 (U.S. Pat. No. 9,683,048), BGB-A317 (U.S. Pat. No. 8,735,553), and MGA012 (MacroGenics, Rockville, Md.). In one embodiment, the anti-human PD-1 monoclonal antibody is pembrolizumab.

In certain embodiments of various methods, pharmaceutical compositions, kits, or uses provided herein, the anti-human PD-1 monoclonal antibody or antigen binding fragment thereof comprises a light chain variable region (V_{L}) complementarily determining region 1 (CDR1), a V_{L} CDR2, and a V_{L} CDR3 comprising amino acid sequences as set forth in SEQ ID NOs:1, 2, and 3, respectively, and a heavy chain variable region (V_{H}) CDR1, a V_{H} CDR2, and a V_{H} CDR3 comprising amino acid sequences as set forth in SEQ ID NOs:6, 7, and 8, respectively.

In some embodiments of various methods, pharmaceutical compositions, kits, or uses provided herein, the anti-human PD-1 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9.

In other embodiments of various methods, pharmaceutical compositions, kits, or uses provided herein, the anti-human PD-1 monoclonal antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10.

| SEQ | | |
|---|---|---|
| ID NO | Description | Sequence |
| 1 | Pembrolizumab, | RASKGVSTSGYSYLH |
| | VL-CDR1 | |
| 2 | Pembrolizumab, | LASYLES |
| | VL-CDR2 | |
| 3 | Pembrolizumab, | QHSRDLPLT |
| | VL-CDR3 | |
| 4 | Pembrolizumab, | EIVLTQSPATLSLSPGERATLSCRASKGVSTSGYSYLH |
| | VL | WYQQKPGQAPRLLIYLASYLESGVPARFSGSGSGTDF |
| | | TLTISSLEPEDFAVYYCQHSRDLPLTFGGGTKVEIK |
| 5 | Pembrolizumab, | EIVLTQSPATLSLSPGERATLSCRASKGVSTSGYSYLH |
| | light chain | WYQQKPGQAPRLLIYLASYLESGVPARFSGSGSGTDF |
| | | TLTISSLEPEDFAVYYCQHSRDLPLTFGGGTKVEIKRT |
| | | VAAPSVFIFPPSDEQLKSGTASVVCLLNNFYPREAKV |
| | | QWKVDNALQSGNSQESVTEQDSKDSTYSLSSTLTLS |
| | | KADYEKHKVYACEVTHQGLSSPVTKSFNRGEC |
| 6 | Pembrolizumab, | NYYMY |
| | VH-CDR1 | |
| 7 | Pembrolizumab, | GINPSNGGTNFNEKFKN |
| | VH-CDR2 | |
| 8 | Pembrolizumab, | RDYRFDMGFDY |
| | VH-CDR3 | |
| 9 | Pembrolizumab, | QVQLVQSGVEVKKPGASVKVSCKASGYTFTNYYMY |
| | VH | WVRQAPGQGLEWMGGINPSNGGTNFNEKFKNRVTL |
| | | TTDSSTTTAYMELKSLQFDDTAVYYCARRDYRFDM |
| | | GFDYWGQGTTVTVSS |
| 10 | Pembrolizumab, | QVQLVQSGVEVKKPGASVKVSCKASGYTFTNYYMY |
| | heavy chain | WVRQAPGQGLEWMGGINPSNGGTNFNEKFKNRVTL |
| | | TTDSSTTTAYMELKSLQFDDTAVYYCARRDYRFDM |
| | | GFDYWGQGTTVTVSSASTKGPSVFPLAPCSRSTSEST |
| | | AALGCLVKDYFPEPVTVSWNSGALTSGVHTFPAVLQ |
| | | SSGLYSLSSVVTVPSSSLGTKTYTCNVDHKPSNTKVD |
| | | KRVESKYGPPCPPCPAPEFLGGPSVFLFPPKPKDTLMI |
| | | SRTPEVTCVVVDVSQEDPEVQFNWYVDGVEVHNAK |
| | | TKPREEQFNSTYRVVSVLTVLHQDWLNGKEYKCKVS |
| | | NKGLPSSIEKTISKAKGQPREPQVYTLPPSQEEMTKN |
| | | QVSLTCLVKGFYPSDIAVEWESNGQPENNYKTTPPVL |
| | | DSDGSFFLYSRLTVDKSRWQEGNVFSCSVMHEALHN |
| | | HYTQKSLSLSLGK |

In another embodiment, the anti-human PD-1 monoclonal antibody is nivolumab. In another embodiment, the anti-human PD-1 monoclonal antibody is cemiplimab. In another embodiment, the anti-human PD-1 monoclonal antibody is sintilimab. In another embodiment, the anti-human PD-1 monoclonal antibody is tiselizumab. In another embodiment, the anti-human PD-1 monoclonal antibody is camrelizumab. In another embodiment, the anti-human PD-1 monoclonal antibody is toripalimab. In yet another embodiment, the anti-human PD-1 monoclonal antibody is pidilizumab. In one embodiment, the anti-human PD-1 monoclonal antibody is AMP-514. In another embodiment, the anti-human PD-1 monoclonal antibody is PDR001. In yet another embodiment, the anti-human PD-1 monoclonal antibody is BGB-A317. In still another embodiment, the anti-human PD-1 monoclonal antibody is MGA012.

In some embodiments, the anti-human PD-1 monoclonal antibody can be any antibody, antigen binding fragment thereof, or variant thereof disclosed in U.S. Pat. Nos. 7,488,802, 7,521,051, 8,008,449, 8,354,509, 8,168,757, WO2004/004771, WO2004/072286, WO2004/056875, US2011/0271358, and WO 2008/156712, the disclosures of which are incorporated by reference herein in their entireties.

Examples of monoclonal antibodies that bind to human PD-L1 that can be used in various methods, kits, and uses described herein are disclosed in U.S. Pat. No. 8,383,796, the disclosures of which are incorporated by reference herein in their entireties. Specific anti-human PD-L1 monoclonal antibodies useful as the PD-1 antagonist in the various methods, kits, and uses described include durvalumab, avelumab, and BMS-936559.

Other PD-1 antagonists useful in various methods, kits, and uses described herein include an immunoadhesion molecule that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342, the disclosures of which are incorporated by reference herein in their entireties. Specific fusion proteins useful as the PD-1 antagonist in various methods, kits, and uses described herein include AMP-224 (also known as B7-DCIg), which is a PD-L2-Fc fusion protein and binds to human PD-1.

In various embodiments, the anti-human PD-1 or anti-human PD-L1 monoclonal antibody or antigen binding fragment thereof comprises a variant of the amino acid sequences of the anti-human PD-1 or anti-human PD-L1 antibodies described herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-human PD-1 or anti-human PD-L1 monoclonal antibody or antigen binding fragment thereof has a V_{L} domain with at least 95%, 90%, 85%, 80%, 75% or 50% sequence homology to one of the V_{L} domains of the anti-human PD-1 or anti-human PD-L1 antibodies described herein, and exhibits specific binding to PD-1 or PD-L1. In another embodiment, the anti-human PD-1 or anti-human PD-L1 monoclonal antibody or antigen binding fragment thereof has a V_{H} domain with at least 95%, 90%, 85%, 80%, 75% or 50% sequence homology to one of the V_{H} domains of the anti-human PD-1 or anti-human PD-L1 antibodies described herein, and exhibits specific binding to PD-1 or PD-L1. In yet another embodiment, the anti-human PD-1 or anti-human PD-L1 monoclonal antibody or antigen binding fragment thereof has a V_{L} domain with at least 95%, 90%, 85%, 80%, 75% or 50% sequence homology to one of the V_{L} domains of the anti-human PD-1 or anti-human PD-L1 antibodies described herein and a V_{H} domain with at least 95%, 90%, 85%, 80%, 75% or 50% sequence homology to one of the V_{H} domains of the anti-human PD-1 or anti-human PD-L1 antibodies described herein, and exhibits specific binding to PD-1 or PD-L1.

In one embodiment, the anti-human PD-1 or anti-human PD-L1 monoclonal antibody or antigen binding fragment thereof has a V_{L} domain having up to 1, 2, 3, 4, 5 or more amino acid substitutions, deletions and/or additions in one of the V_{L} domains of the anti-human PD-1 or anti-human PD-L1 antibodies described herein, and exhibits specific binding to PD-1 or PD-L1. In another embodiment, the anti-human PD-1 or anti-human PD-L1 monoclonal antibody or antigen binding fragment thereof has a V_{H} domain having up to 1, 2, 3, 4, 5 or more amino acid substitutions, deletions, and/or additions in one of the V_{H} domains of the anti-human PD-1 or anti-human PD-L1 antibodies described herein, and exhibits specific binding to PD-1 or PD-L1. In yet another embodiment, the anti-human PD-1 or anti-human PD-L1 monoclonal antibody or antigen binding fragment thereof has a V_{L} domain having up to 1, 2, 3, 4, 5 or more amino acid substitutions, deletions, and/or additions in one of the V_{L} domains of the anti-human PD-1 or anti-human PD-L1 antibodies described herein and a V_{H} domain having up to 1, 2, 3, 4, or more amino acid substitutions, deletions, and/or additions in one of the V_{H} domains of the anti-human PD-1 or anti-human PD-L1 antibodies described herein, and exhibits specific binding to PD-1 or PD-L1.

In various embodiments, the anti-human PD-1 or anti-human PD-L1 monoclonal antibody or antigen binding fragment thereof is selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-human PD-1 or anti-human PD-L1 monoclonal antibodies and antigen binding fragments thereof disclosed herein will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3. M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

In some embodiments, the anti-human PD-1 or anti-human PD-L1 monoclonal antibody is a human antibody. In other embodiments, the anti-human PD-1 or anti-human PD-L1 monoclonal antibody is a humanized antibody.

In some embodiments, the light chain of the anti-human PD-1 or anti-human PD-L1 monoclonal antibody has a human kappa backbone. In other embodiments, the light chain of the anti-human PD-1 or anti-human PD-L1 monoclonal antibody has a human lambda backbone.

In some embodiments, the heavy chain of the anti-human PD-1 or anti-human PD-L1 monoclonal antibody has a human IgG1 backbone. In other embodiments, the heavy chain of the anti-human PD-1 or anti-human PD-L1 monoclonal antibody has a human IgG2 backbone. In yet other embodiments, the heavy chain of the anti-human PD-1 or anti-human PD-L1 monoclonal antibody has a human IgG3 backbone. In still other embodiments, the heavy chain of the anti-human PD-1 or anti-human PD-L1 monoclonal antibody has a human IgG4 backbone.

In some embodiments, the heavy chain of the anti-human PD-1 or anti-human PD-L1 monoclonal antibody has a human IgG1 variant backbone. In other embodiments, the heavy chain of the anti-human PD-1 or anti-human PD-L1 monoclonal antibody has a human IgG2 variant backbone. In yet other embodiments, the heavy chain of the anti-human PD-1 or anti-human PD-L1 monoclonal antibody has a human IgG3 variant backbone. In still other embodiments, the heavy chain of the anti-human PD-1 or anti-human PD-L1 monoclonal antibody has a human IgG4 variant (e.g., IgG4 with S228P mutation) backbone.

### 3. HIF-2α Inhibitors

Also provided herein are HIF-2α inhibitors that can be used in the various methods, kits, and uses disclosed herein, including any chemical compound or biological molecule that inhibits the activity of HIF-2α.

In some embodiments, the HIF-2α inhibitor is belzutifan, or a pharmaceutically acceptable salt thereof, which is also known as MK-6482, PT2977, 3-[(1S,2S,3R)-2,3-difluoro-1-hydroxy-7-methylsulfonyl-indan-4-yl]oxy-5-fluoro-benzonitrile, and 3-[[(1S,2S,3R)-2,3-difluoro-2,3-dihydro-1-hydroxy-7-(methylsulfonyl)-1H-inden-4-yl]oxy]-5-fluorobenzonitrile and has the following chemical structure:

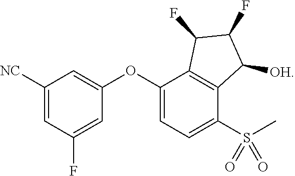 [SMILES: CS(=O)(=O)c1ccc(Oc2cc(F)cc(C#N)c2)c2c1[C@H](O)[C@H](F)[C@@H]2F]

Belzutifan and its synthesis are described in U.S. Pat. No. 9,969,689, which is hereby incorporated by reference in its entirety. Belzutifan as a potential treatment for clear cell renal cell carcinoma is described in Rui Xu et al., J. Med. Chem. 2019, 62, 6876-6893, which is hereby incorporated by reference in its entirety. Combinations of a PD-1/CTLA-4 inhibitor with a HIF-2α inhibitor for the treatment of melanoma, RCC or CRC are described in U.S. Pat. No. 10,335,388, which is hereby incorporated by reference in its entirety. U.S. Publication 2018-0042884 describes the treatment of glioblastoma with a HIF-2α inhibitor, and is hereby incorporated by reference in its entirety. Oral formulations of belzutifan are described in International Application No. PCT/US2019/57725, which was filed on Oct. 23, 2019, and is hereby incorporated by reference in its entirety.

### 4. Lenvatinib

Also provided herein is lenvatinib, which is a multiple RTK (multi-RTK) inhibitor that selectively inhibits the kinase activities of VEGF receptors.

Lenvatinib, which is also known as LENVIMA®, Eisai Inc., Woodcliff Lake, N.J. and 4-[3-chloro-4-(cyclopropylaminocarbonyl)aminophenoxy]-7-methoxy-6-quinolinecarboxamide, has the following chemical structure:

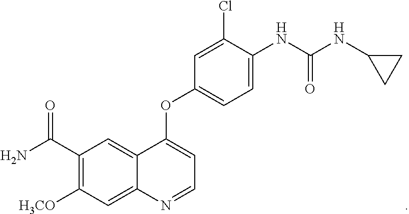 [SMILES: COc1cc2nccc(Oc3ccc(NC(=O)NC4CC4)c(Cl)c3)c2cc1C(N)=O]

Levatinib, its synthesis and uses are described in U.S. Pat. Nos. 7,253,286; 7,612,208; 9,006,256; 10,259,791; and 10,407,393 which are hereby incorporated by reference in their entirety.

### 5. Methods of Treating Cancer or Von-Hippel Lindau Disease, Using a Combination of a PD-1 Antagonist, an HIF-2α Inhibitor, and Lenvatinib or a Pharmaceutically Acceptable Salt Thereof

In another aspect, provided herein are methods of treating cancer (e.g., RCC) or von-Hippel Lindau disease, using a combination of a PD-1 antagonist, a HIF-2α inhibitor, and lenvatinib or a pharmaceutically acceptable salt thereof described.

In some embodiments, the PD-1 antagonist is an anti-PD-1 antibody or antigen binding fragment thereof.

In certain embodiments, the method of treating cancer or von-Hippel Lindau disease comprises administering to a human patient in need thereof:

(a) a PD-1 antagonist;

(b) a HIF-2α inhibitor; and

(c) lenvatinib, or a pharmaceutically acceptable salt thereof.

In some embodiments, the cancer is selected from the group consisting of bladder cancer, breast cancer, non-small cell lung cancer (NSCLC), colorectal cancer (CRC), renal cell carcinoma (RCC), hepatocellular carcinoma (HCC), pancreatic cancer and melanoma.

In certain embodiments, the cancer is metastatic. In some embodiments, the cancer is relapsed. In other embodiments, the cancer is refractory. In yet other embodiments, the cancer is relapsed and refractory.

In one embodiment, the cancer is bladder cancer. In another embodiment, the cancer is breast cancer. In yet another embodiment, the cancer is NSCLC. In still another embodiment, the cancer is CRC. In one embodiment, the cancer is RCC. In another embodiment, the cancer is HCC. In yet another embodiment, the cancer is pancreatic cancer. In yet another embodiment, the cancer is melanoma.

In one embodiment, the cancer is advanced RCC. In another embodiment, the RCC is advanced RCC with clear cell component (ccRCC). In yet another embodiment, the cancer is metastatic RCC. In yet another embodiment, the cancer is relapsed RCC. In still another embodiment, the cancer is refractory RCC. In yet still another embodiment, the cancer is relapsed and refractory RCC.

In one embodiment, the human patient has not received prior systemic treatment for advanced disease. In a class of the embodiment, the human patient has not received prior systemic treatment for advanced RCC.

In certain embodiments, provided herein is a method of treating RCC, comprising administering to a human patient in need thereof:

(a) a PD-1 antagonist;

(b) a HIF-2α inhibitor; and

(c) lenvatinib, or a pharmaceutically acceptable salt thereof.

In some embodiments, provided herein is a method of treating advanced RCC, comprising administering to a human patient in need thereof:

(a) a PD-1 antagonist;

(b) a HIF-2α inhibitor; and

(c) lenvatinib, or a pharmaceutically acceptable salt thereof.

In some embodiments, provided herein is a method of treating advanced RCC with clear cell component, comprising administering to a human patient in need thereof:

(a) a PD-1 antagonist;

(b) a HIF-2α inhibitor; and

(c) lenvatinib, or a pharmaceutically acceptable salt thereof.

In other embodiments, provided herein is a method of treating metastatic RCC, comprising administering to a human patient in need thereof:

(a) a PD-1 antagonist;

(b) a HIF-2α inhibitor; and

(c) lenvatinib, or a pharmaceutically acceptable salt thereof.

In yet other embodiments, provided herein is a method of treating relapsed RCC, comprising administering to a human patient in need thereof:

(a) a PD-1 antagonist;

(b) a HIF-2α inhibitor; and

(c) lenvatinib, or a pharmaceutically acceptable salt thereof.

In yet still other embodiments, provided herein is a method of treating refractory RCC, comprising administering to a human patient in need thereof:

(a) a PD-1 antagonist;

(b) a HIF-2α inhibitor; and

(c) lenvatinib, or a pharmaceutically acceptable salt thereof.

In other embodiments, provided herein is a method of treating relapsed and refractory RCC, comprising administering to a human patient in need thereof:

(a) a PD-1 antagonist;

(b) a HIF-2α inhibitor; and

(c) lenvatinib, or a pharmaceutically acceptable salt thereof.

In other embodiments, provided herein is a method of treating pancreatic cancer, comprising administering to a human patient in need thereof:

(a) a PD-1 antagonist;

(b) a HIF-2α inhibitor; and

(c) lenvatinib, or a pharmaceutically acceptable salt thereof.

In certain embodiments, the method of treating cancer comprises administering to a human patient in need thereof:
(a) a PD-1 antagonist;
(b) belzutifan, or a pharmaceutically acceptable salt thereof; and
(c) lenvatinib, or a pharmaceutically acceptable salt thereof.

In certain embodiments, the PD-1 antagonist is an anti-human PD-1 monoclonal antibody or antigen binding fragment thereof. In some embodiments, the anti-human PD-1 monoclonal antibody is a human antibody. In other embodiments, the anti-human PD-1 monoclonal antibody is a humanized antibody.

In certain embodiments, the PD-1 antagonist is an anti-human PD-L1 monoclonal antibody or antigen binding fragment thereof. In some embodiments, the anti-human PD-L1 monoclonal antibody is a human antibody. In other embodiments, the anti-human PD-L1 monoclonal antibody is a humanized antibody.

Thus, in certain embodiments, provided herein is a method for treating cancer, comprising administering to a human patient in need thereof:
(a) a human or humanized anti-human PD-1 monoclonal antibody or antigen binding fragment thereof
(b) belzutifan, or a pharmaceutically acceptable salt thereof; and
(c) lenvatinib, or a pharmaceutically acceptable salt thereof.

In some embodiments, provided herein is a method for treating cancer, comprising administering to a human patient in need thereof:
(a) a human anti-human PD-1 monoclonal antibody or antigen binding fragment thereof;
(b) belzutifan, or a pharmaceutically acceptable salt thereof; and
(c) lenvatinib, or a pharmaceutically acceptable salt thereof.

In other embodiments, provided herein is a method for treating cancer, comprising administering to a human patient in need thereof:
(a) a humanized anti-human PD-1 monoclonal antibody or antigen binding fragment thereof
(b) belzutifan, or a pharmaceutically acceptable salt thereof; and
(c) lenvatinib, or a pharmaceutically acceptable salt thereof.

In one embodiment of various methods provided herein, the anti-human PD-1 monoclonal antibody or antigen binding fragment thereof is pembrolizumab.

In another embodiment of various methods provided herein, the anti-human PD-1 monoclonal antibody or antigen binding fragment thereof is nivolumab.

In another embodiment of various methods provided herein, the anti-human PD-1 monoclonal antibody or antigen binding fragment thereof is cemiplimab.

Thus, in one specific embodiment of various methods provided herein, the method for treating cancer comprises administering to a human patient in need thereof:

(a) pembrolizumab;

(b) belzutifan, or a pharmaceutically acceptable salt thereof; and

(c) lenvatinib, or a pharmaceutically acceptable salt thereof.

In one specific embodiment of various methods provided herein, the method for treating cancer comprises administering to a human patient in need thereof:

(a) nivolumab;

(b) belzutifan, or a pharmaceutically acceptable salt thereof; and

(c) lenvatinib, or a pharmaceutically acceptable salt thereof.

In one specific embodiment of various methods provided herein, the method for treating cancer comprises administering to a human patient in need thereof:

(a) cemiplimab;

(b) belzutifan, or a pharmaceutically acceptable salt thereof; and

(c) lenvatinib, or a pharmaceutically acceptable salt thereof.

In one specific embodiment of various methods provided herein, the method for treating RCC comprises administering to a human patient in need thereof:

(a) pembrolizumab;

(b) belzutifan, or a pharmaceutically acceptable salt thereof; and

(c) lenvatinib, or a pharmaceutically acceptable salt thereof.

In one specific embodiment of various methods provided herein, the method for treating RCC comprises administering to a human patient in need thereof:

(a) nivolumab;

(b) belzutifan, or a pharmaceutically acceptable salt thereof; and

(c) lenvatinib, or a pharmaceutically acceptable salt thereof.

In one specific embodiment of various methods provided herein, the method for treating RCC comprises administering to a human patient in need thereof:

(a) cemiplimab;

(b) belzutifan, or a pharmaceutically acceptable salt thereof; and

(c) lenvatinib, or a pharmaceutically acceptable salt thereof.

In one embodiment, the RCC is advanced RCC. In another embodiment, the RCC is advanced RCC with clear cell component. In yet another embodiment, the RCC is metastatic RCC. In yet another embodiment, the RCC is relapsed RCC. In still another embodiment, the RCC is refractory RCC. In yet still another embodiment, the RCC is relapsed and refractory RCC.

In one embodiment, the present invention provides a method of treating von Hippel-Lindau (VHL) disease, comprising administering to a human patient in need thereof:

(a) a PD-1 antagonist (e.g., pembrolizumab);

(b) a HIF-2α inhibitor (e.g., belzutifan, or a pharmaceutically acceptable salt thereof); and

(c) lenvatinib, or a pharmaceutically acceptable salt thereof.

### 6. Dosing and Administration

Further provided herein are dosing regimens and routes of administration for treating cancer (e.g., RCC) using a combination of a PD-1 antagonist (e.g., an anti-PD-1 monoclonal antibody or antigen binding fragment thereof), a HIF-2α inhibitor, and a multi-RTK inhibitor (e.g., lenvatinib or a pharmaceutically acceptable salt thereof).

The anti-PD-1 monoclonal antibody or antigen binding fragment thereof, the HIF-2a inhibitor, or lenvatinib or a pharmaceutically acceptable salt thereof disclosed herein may be administered by doses administered, e.g., daily, 1-7 times per week, weekly, bi-weekly, tri-weekly, every four weeks, every five weeks, every 6 weeks, monthly, bimonthly, quarterly, semiannually, annually, etc. Doses may be administered, e.g., intravenously, subcutaneously, topically, orally, nasally, rectally, intramuscular, intracerebrally, intraspinally, or by inhalation. In certain embodiments, the closes are administered intravenously. In certain embodiments, the doses are administered subcutaneously. In certain embodiments, the doses are administered orally. A total dose for a treatment interval is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more. Doses may also be provided to achieve a pre-determined target concentration of the antibody (e.g., anti-PD-1 antibody) or antigen binding fragment thereof in the subject's serum, such as 0.1, 0.3, 1, 3, 10, 30, 100, 300 or more.

In some embodiments, the anti-PD-1 monoclonal antibody or antigen binding fragment thereof is administered subcutaneously or intravenously, on a weekly, biweekly, triweekly, every 4 weeks, every 5 weeks, every 6 weeks, monthly, bimonthly, or quarterly basis at 10, 20, 50, 80, 100, 200, 300, 400, 500, 1000 or 2500 mg/subject. In some specific methods, the dose of the anti-PD-1 monoclonal antibody or antigen binding fragment thereof is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, or from about 2.0 mg/kg to about 4.0 mg/kg. In some specific methods, the dose of the anti-PD-1 monoclonal antibody or antigen binding fragment thereof is from about 10 mg to about 500 mg, from about 25 mg to about 500 mg, from about 50 mg to about 500 mg, from about 100 mg to about 500 mg, from about 200 mg to about 500 mg, from about 150 mg to about 250 mg, from about 175 mg to about 250 mg, from about 200 mg to about 250 mg, from about 150 mg to about 240 mg, from about 175 mg to about 240 mg, or from about 200 mg to about 240 mg. In some embodiments, the dose of the anti-PD-1 monoclonal antibody or antigen binding fragment thereof is 50 mg, 75 mg, 100 mg, 125 mg, 150 mg, 175 mg, 200 mg, 225 mg, 240 mg, 250 mg, 300 mg, 400 mg, or 500 mg.

In some embodiments of various methods described herein, the anti-human PD-1 monoclonal antibody or antigen binding fragment thereof is pembrolizumab, the human patient is administered 200 mg, 240 mg, or 2 mg/kg pembrolizumab, and pembrolizumab is administered once every three weeks. In one embodiment, the human patient is administered 200 mg pembrolizumab once every three weeks. In one embodiment, the human patient is administered 240 mg pembrolizumab once every three weeks. In one embodiment, the human patient is administered 2 mg/kg pembrolizumab once every three weeks.

In certain embodiments of various methods described herein, the anti-human PD-1 monoclonal antibody or antigen binding fragment thereof is pembrolizumab, the human patient is administered 400 mg pembrolizumab, and pembrolizumab is administered once every six weeks.

In other embodiments of various methods described herein, the anti-human PD-1 monoclonal antibody or antigen binding fragment thereof is nivolumab, the human patient is administered 240 mg or 3 mg/kg nivolumab, and nivolumab is administered once every two weeks. In one specific embodiment, the human patient is administered 240 mg nivolumab once every two weeks. In one specific embodiment, the human patient is administered 3 mg/kg nivolumab once every two weeks. In other embodiments of various methods described herein, the anti-human PD-1 monoclonal antibody or antigen binding fragment thereof is nivolumab, the human patient is administered 480 mg nivolumab, and nivolumab is administered once every four weeks.

In yet other embodiments of various methods described herein, the anti-human PD-1 monoclonal antibody or antigen binding fragment thereof is cemiplimab, the human patient is administered 350 mg cemiplimab, and cemiplimab is administered once every three weeks.

In still other embodiments of various methods described herein, the HIF-2α inhibitor is belzutifan, or a pharmaceutically acceptable salt thereof, and the human patient is administered from 40 to 120 mg once-daily. In still other embodiments of various methods described herein, 40, 80 or 120 mg of belzutifan, or a pharmaceutically acceptable salt thereof is administered once-daily. In one specific embodiment, the human patient is administered 40 mg of belzutifan, or a pharmaceutically acceptable salt thereof once-daily. In one specific embodiment, the human patient is administered 80 mg of belzutifan, or a pharmaceutically acceptable salt thereof once-daily. In one specific embodiment, the human patient is administered 120 mg of belzutifan, or a pharmaceutically acceptable salt thereof once-daily.

In certain embodiments, lenvatinib or a pharmaceutically acceptable salt thereof is administered orally. In some embodiments, lenvatinib or a pharmaceutically acceptable salt thereof is administered at a daily dose of 8, 10, 12, 14, 18, 20, or 24 mg, each as lenvatinib.

Thus, in some embodiments of various methods provided herein, the human patient is administered:
(a) 200 mg, 240 mg, or 2 mg/kg pembrolizumab;
(b) 40, 80, or 120 mg belzutifan; and
(c) 8, 10, 12, 14, 18, 20, or 24 mg lenvatinib;
wherein (a) is administered once every three weeks; and wherein (b) and (c) are administered daily.

In certain embodiments of various methods provided herein, the human patient is administered:
(a) 200 mg pembrolizumab;
(b) 120 mg belzutifan; and
(c) 20 mg lenvatinib;
wherein (a) is administered once every three weeks; and wherein (b) and (c) are administered daily.

In certain embodiments of various methods provided herein, the human patient is administered:
(a) 240 mg pembrolizumab;
(b) 120 mg belzutifan; and
(c) 20 mg lenvatinib;
wherein (a) is administered once every three weeks; and wherein (b) and (c) are administered daily.

In certain embodiments of various methods provided herein, the human patient is administered:
(a) 2 mg/kg pembrolizumab;
(b) 120 mg belzutifan; and
(c) 20 mg lenvatinib;
wherein (a) is administered once every three weeks; and wherein (b) and (c) are administered daily.

In certain embodiments of various methods provided herein, the human patient is
(a) 400 mg pembrolizumab;
(b) 120 mg belzutifan; and
(c) 20 mg lenvatinib;
wherein (a) is administered once every six weeks; and wherein (b) and (c) are administered daily.

In certain embodiments of various methods provided herein, the human patient is administered:
(a) 400 mg pembrolizumab;
(b) 120 mg belzutifan; and
(c) 14 mg lenvatinib;
wherein (a) is administered once every six weeks; and wherein (b) and (c) are administered daily.

In certain embodiments of various methods provided herein, the human patient is administered:
(a) 400 mg pembrolizumab;
(b) 120 mg belzutifan; and
(c) 10 mg lenvatinib;
wherein (a) is administered once every six weeks; and wherein (b) and (c) are administered daily.

In certain embodiments of various methods provided herein, the human patient is administered:
(a) 400 mg pembrolizumab;
(b) 80 mg belzutifan; and
(c) 10 mg lenvatinib;
wherein (a) is administered once every six weeks; and wherein (b) and (c) are administered daily.

In certain embodiments of various methods provided herein, the human patient is administered:
(a) 200 mg pembrolizumab;
(b) 120 mg belzutifan; and
(c) 20 mg lenvatinib;
wherein (a) is administered once every three weeks; and wherein (b) and (c) are administered daily.

In certain embodiments of various methods provided herein, the human patient is administered:
(a) 240 mg pembrolizumab;
(b) 120 mg belzutifan; and
(c) 20 mg lenvatinib;
wherein (a) is administered once every three weeks; and wherein (b) and (c) are administered daily.

In certain embodiments of various methods provided herein, the human patient is administered:
(a) 2 mg/kg pembrolizumab;
(b) 120 mg belzutifan; and
(c) 20 mg lenvatinib;
wherein (a) is administered once every three weeks; and wherein (b) and (c) are administered daily.

In certain embodiments of various methods provided herein, the human patient is administered:
(a) 400 mg pembrolizumab;
(b) 120 mg belzutifan; and
(c) 20 mg lenvatinib;
wherein (a) is administered once every six weeks; and wherein (b) and (c) are administered daily.

In certain embodiments, the human patient has not received prior systemic treatment for advanced disease.

In some embodiments, at least one of the therapeutic agents (e.g., the anti-PD-1 monoclonal antibody or binding fragment thereof, the HIF-2α inhibitor, or lenvatinib) in the combination therapy is administered using the same dosage regimen (dose, frequency, and duration of treatment) that is typically employed when the agent is used as monotherapy for treating the same condition. In other embodiments, the patient receives a lower total amount of at least one of the therapeutic agents (e.g., the anti-PD-1 monoclonal antibody or binding fragment thereof, the HIF-2α inhibitor, or lenvatinib) in the combination therapy than when the agent is used as monotherapy, e.g., smaller doses, less frequent doses, and/or shorter treatment duration.

A combination therapy disclosed herein may be used prior to or following surgery to remove a tumor and may be used prior to, during, or after radiation treatment.

In some embodiments, a combination therapy disclosed herein is administered to a patient who has not previously been treated with a biotherapeutic or chemotherapeutic agent, i.e., is treatment-naïve. In other embodiments, the combination therapy is administered to a patient who failed to achieve a sustained response after prior therapy with the biotherapeutic or chemotherapeutic agent, i.e., is treatment-experienced.

The therapeutic combination disclosed herein may be used in combination with one or more other active agents, including but not limited to, other anti-cancer agents that are used in the prevention, treatment, control, amelioration, or reduction of risk of a particular disease or condition (e.g., cancer). Such other active agents may be administered, by a route and in an amount commonly used therefor, contemporaneously or sequentially with one or more of the therapeutic agents in the combinations disclosed herein.

The one or more additional active agents may be co-administered with the anti-PD-1 monoclonal antibody or antigen binding fragment thereof, the HIF-2α inhibitor, or lenvatinib or a pharmaceutically acceptable salt thereof. The additional active agent(s) can be administered in a single dosage form with one or more co-administered agent selected from the anti-PD-1 monoclonal antibody or antigen binding fragment thereof, the HIF-2α inhibitor, and lenvatinib or a pharmaceutically acceptable salt thereof. The additional active agent(s) can also be administered in separate dosage form(s) from the dosage forms containing the anti-PD-1 monoclonal antibody or antigen binding fragment thereof, the HIF-2α inhibitor, or lenvatinib or a pharmaceutically acceptable salt thereof.

### 7. Kits

In still another aspect, provided herein are kits comprising the therapeutic agents disclosed herein (e.g., a PD-1 antagonist, a HIF-2α inhibitor, and lenvatinib) or pharmaceutical compositions thereof, packaged into suitable packaging material. A kit optionally includes a label or packaging insert that include a description of the components or instructions for use in vitro, in vivo, or ex vivo, of the components therein.

In some embodiments, the kit comprises
(a) a PD-1 antagonist;
(b) a HIF-2α inhibitor; and
(c) lenvatinib, or a pharmaceutically acceptable salt thereof.

In certain embodiments, the kit further comprises instructions for administering to a human patient the PD-1 antagonist, the HIF-2α inhibitor, and lenvatinib or a pharmaceutically acceptable salt thereof.

In some embodiments, the PD-1 antagonist is an anti-PD-1 monoclonal antibody or antigen-binding fragment thereof. In some embodiments, the PD-1 antagonist is an anti-PD-L1 monoclonal antibody or antigen-binding fragment thereof.

In one embodiment, the kit comprises: (a) one or more dosages of an anti-PD-1 monoclonal antibody or antigen binding fragment thereof (b) one or more dosages of a HIF-2α inhibitor; (c) one or more dosages of lenvatinib or a pharmaceutically acceptable salt thereof; and (d) instructions for administering to a human patient the anti-human PD-1 monoclonal antibody or antigen binding fragment thereof, the HIF-2α inhibitor, and lenvatinib or a pharmaceutically acceptable salt thereof.

In some embodiments, the anti-PD-1 monoclonal antibody or antigen binding fragment thereof is pembrolizumab. In some embodiments, the anti-PD-1 monoclonal antibody or antigen binding fragment thereof is nivolumab. In some embodiments, the anti-PD-1 monoclonal antibody or antigen binding fragment thereof is cemiplimab.

The dosages for the anti-PD-1 monoclonal antibody, the HIF-2α inhibitor, or lenvatinib or a pharmaceutically acceptable salt thereof can be used in various kits herein. In some embodiments, a kit comprises dosages of each component sufficient for a certain period of treatment (e.g., 3, 6, 12, or 24 weeks, etc.). For example, a kit can comprise a dosage of 200 mg pembrolizumab, 21 dosages of 120 mg belzutifan, or a pharmaceutically acceptable salt thereof, and 21 dosages of 20 mg lenvatinib (or equivalent amount of a pharmaceutically acceptable salt of lenvatinib), which are sufficient for a 3-week treatment. Or, a kit can also comprise a dosage of 400 mg pembrolizumab, 42 dosages of 120 mg belzutifan, or a pharmaceutically acceptable salt thereof, and 42 dosages of 20 mg lenvatinib (or equivalent amount of a pharmaceutically acceptable salt of lenvatinib), which are sufficient for a 6-week treatment.

In some embodiments, the kit comprises means for separately retaining the components, such as a container, divided bottle, or divided foil packet. A kit of this disclosure can be used for administration of different dosage forms, for example, oral and parenteral, for administration of the separate compositions at different dosage intervals, or for titration of the separate compositions against one another.

### 8. Uses of a Therapeutic Combination for Treating Cancer or Von-Hippel Lindau Disease

In still another aspect, provided herein are uses of a therapeutic combination for treating cancer (e.g., RCC) or von-Hippel Lindau disease in a human patient, wherein the therapeutic combination comprises:
(a) a PD-1 antagonist;
(b) a HIF-2α inhibitor; and
(c) lenvatinib, or a pharmaceutically acceptable salt thereof.

In some embodiments, the cancer is selected from the group consisting of bladder cancer, breast cancer, non-small cell lung cancer (NSCLC), colorectal cancer (CRC), renal cell carcinoma (RCC), hepatocellular carcinoma (HCC), and melanoma.

In certain embodiments, the cancer is metastatic. In some embodiments, the cancer is relapsed. In other embodiments, the cancer is refractory. In yet other embodiments, the cancer is relapsed and refractory.

In one embodiment, the cancer is bladder cancer. In another embodiment, the cancer is breast cancer. In yet another embodiment, the cancer is NSCLC. In still another embodiment, the cancer is CRC. In one embodiment, the cancer is RCC. In another embodiment, the cancer is HCC. In yet another embodiment, the cancer is melanoma.

In one embodiment, the cancer is advanced RCC. In another embodiment, the cancer is advanced RCC with clear cell component. In yet another embodiment, the cancer is metastatic RCC. In yet another embodiment, the cancer is relapsed RCC. In still another embodiment, the cancer is refractory RCC. In yet still another embodiment, the cancer is relapsed and refractory RCC.

In one embodiment, provided herein is use of a therapeutic combination for treating RCC in a human patient, wherein the therapeutic combination comprises:
(a) a PD-1 antagonist;
(b) a HIF-2α inhibitor; and
(c) lenvatinib, or a pharmaceutically acceptable salt thereof.

In some embodiments, provided herein is use of a therapeutic combination for treating advanced RCC in a human patient, wherein the therapeutic combination comprises:

(a) a PD-1 antagonist;

(b) a HIF-2α inhibitor; and

(c) lenvatinib, or a pharmaceutically acceptable salt thereof.

In some embodiments, provided herein is use of a therapeutic combination for treating advanced RCC with clear cell component in a human patient, wherein the therapeutic combination comprises:

(a) a PD-1 antagonist;

(b) a HIF-2α inhibitor; and

(c) lenvatinib, or a pharmaceutically acceptable salt thereof.

In other embodiments, provided herein is use of a therapeutic combination for treating metastatic RCC in a human patient, wherein the therapeutic combination comprises:

(a) a PD-1 antagonist;

(b) a HIF-2α inhibitor; and

(c) lenvatinib, or a pharmaceutically acceptable salt thereof.

In yet other embodiments, provided herein is use of a therapeutic combination for treating relapsed RCC in a human patient, wherein the therapeutic combination comprises:

(a) a PD-1 antagonist;

(b) a HIF-2α inhibitor; and

(c) lenvatinib, or a pharmaceutically acceptable salt thereof.

In yet still other embodiments, provided herein is use of a therapeutic combination for treating refractory RCC in a human patient, wherein the therapeutic combination comprises:

(a) a PD-1 antagonist;

(b) a HIF-2α inhibitor; and

(c) lenvatinib, or a pharmaceutically acceptable salt thereof.

In other embodiments, provided herein is use of a therapeutic combination for treating relapsed and refractory RCC in a human patient, wherein the therapeutic combination comprises:

(a) a PD-1 antagonist;

(b) a HIF-2α inhibitor; and

(c) lenvatinib, or a pharmaceutically acceptable salt thereof.

In still other embodiments, provided herein is use of a therapeutic combination for treating cancer, wherein the therapeutic combination comprises:
(a) a PD-1 antagonist;
(b) belzutifan, or a pharmaceutically acceptable salt thereof; and
(c) lenvatinib, or a pharmaceutically acceptable salt thereof.

In certain embodiments, the PD-1 antagonist is an anti-human PD-1 monoclonal antibody or antigen binding fragment thereof. In some embodiments, the anti-human PD-1 monoclonal antibody is a human antibody. In other embodiments, the anti-human PD-1 monoclonal antibody is a humanized antibody.

In certain embodiments, the PD-1 antagonist is an anti-human PD-L1 monoclonal antibody or antigen binding fragment thereof. In some embodiments, the anti-human PD-L1 monoclonal antibody is a human antibody. In other embodiments, the anti-human PD-L1 monoclonal antibody is a humanized antibody.

In certain embodiments, the HIF-2α inhibitor is belzutifan, or a pharmaceutically acceptable salt thereof.

Thus, in certain embodiments, provided herein is use of a therapeutic combination for treating cancer, wherein the therapeutic combination comprises:
(a) a human or humanized anti-human PD-1 monoclonal antibody or antigen binding fragment thereof;
(b) belzutifan, or a pharmaceutically acceptable salt thereof; and
(c) lenvatinib, or a pharmaceutically acceptable salt thereof.

In some embodiments, provided herein is use of a therapeutic combination for treating cancer, wherein the therapeutic combination comprises:
(a) a human anti-human PD-1 monoclonal antibody or antigen binding fragment thereof;
(b) belzutifan, or a pharmaceutically acceptable salt thereof; and
(c) lenvatinib, or a pharmaceutically acceptable salt thereof.

In other embodiments, provided herein is use of a therapeutic combination for treating cancer, wherein the therapeutic combination comprises:
(a) a humanized anti-human PD-1 monoclonal antibody or antigen binding fragment thereof;
(b) belzutifan, or a pharmaceutically acceptable salt thereof; and
(c) lenvatinib, or a pharmaceutically acceptable salt thereof.

In some embodiments of various uses provided herein, the anti-PD-1 monoclonal antibody or antigen binding fragment thereof is pembrolizumab. In some embodiments of various uses provided herein, the anti-PD-1 monoclonal antibody or antigen binding fragment thereof is nivolumab. In some embodiments of various uses provided herein, the anti-PD-1 monoclonal antibody or antigen binding fragment thereof is cemiplimab.

Thus, in one specific embodiment, provided herein is use of a therapeutic combination for treating cancer, wherein the therapeutic combination comprises:

(a) pembrolizumab;

(b) belzutifan, or a pharmaceutically acceptable salt thereof; and

(c) lenvatinib, or a pharmaceutically acceptable salt thereof.

In one specific embodiment, provided herein is use of a therapeutic combination for treating cancer, wherein the therapeutic combination comprises:

(a) nivolumab;

(b) belzutifan, or a pharmaceutically acceptable salt thereof; and

(c) lenvatinib, or a pharmaceutically acceptable salt thereof.

In one specific embodiment, provided herein is use of a therapeutic combination for treating cancer, wherein the therapeutic combination comprises:

(a) cemiplimab;

(b) belzutifan, or a pharmaceutically acceptable salt thereof; and

(c) lenvatinib, or a pharmaceutically acceptable salt thereof.

In one specific embodiment, provided herein is use of a therapeutic combination for treating RCC, wherein the therapeutic combination comprises:

(a) pembrolizumab;

(b) belzutifan, or a pharmaceutically acceptable salt thereof; and

(c) lenvatinib, or a pharmaceutically acceptable salt thereof.

In one specific embodiment, provided herein is use of a therapeutic combination for treating RCC, wherein the therapeutic combination comprises:

(a) nivolumab;

(b) belzutifan, or a pharmaceutically acceptable salt thereof; and

(c) lenvatinib, or a pharmaceutically acceptable salt thereof.

In one specific embodiment, provided herein is use of a therapeutic combination for treating RCC, wherein the therapeutic combination comprises:

(a) cemiplimab;

(b) belzutifan, or a pharmaceutically acceptable salt thereof; and

(c) lenvatinib, or a pharmaceutically acceptable salt thereof.

In one embodiment, the human patient has not received prior systemic treatment for advanced disease.

In one embodiment, the RCC is advanced RCC. In another embodiment, the RCC is advanced RCC with clear cell component. In yet another embodiment, the RCC is metastatic RCC. In yet another embodiment, the RCC is relapsed RCC. In still another embodiment, the RCC is refractory RCC. In yet still another embodiment, the RCC is relapsed and refractory RCC.

A number of embodiments of the invention have been described. It will be understood that various modifications may be made without departing from the spirit and scope of the invention. It will be further understood that each embodiment may be combined with one or more other embodiments, to the extent that such a combination is consistent with the description of the embodiments.

### I. EXAMPLES

The examples in this section (section VI) are offered by way of illustration, and not by way of limitation.

### Example 1

Clinical Trial of Administering an Anti-PD-1 Antibody in Combination with Belzutifan (MK-6482) and Lenvatinib (Experimental Arm A4) in Patients with First-Line (1L) Advanced RCC with Clear Cell Component (ccRCC).

A total of 34 healthy volunteers and 185 patients had been treated with MK-6482 in five ongoing clinical studies.

In an ongoing randomized, single-dose, 2 period, 2 sequence cross-over Phase 1 study in 16 healthy female adult volunteers, the effect of food on the PK of a single dose of 120 mg MK-6482 was investigated. This study indicated that a high fat, high calorie meal did not affect the extent of MK-6482 exposure but lowered the maximum plasma MK-6482 concentration by approximately 35% and delayed time to peak MK-6482 exposure with a median difference (Fed-Fasted) of 2 hours. These data are not considered to be clinically meaningful and support dosing of MK-6482 with or without food. The most common adverse event (AE) reported was headache (12.5%).

A FIH Phase 1 study designed to assess the tolerability, safety, PK, and PD properties of MK-6482 in participants with various advanced solid tumors is ongoing. As of 6 Sep. 2019 a total of 104 participants had been enrolled, including 43 participants with various advanced solid tumors in the dose-escalation portion (Part 1A) ranging from 20 to 240 mg QD and 120 mg BID. The MTD was not reached and 2 treatment-related DLTs were observed: 1 Grade 4 event of thrombocytopenia in the 240 mg QD cohort, and 1 Grade 3 event of hypoxia in the 120 mg BID cohort. The 120 mg QD dose was selected for further clinical development based on favorable PK, pharmacodynamic, and safety findings. 52 additional participants with advanced RCC were treated in an expansion cohort (Part 1B) at the clinical dose of 120 mg QD. In the combined dose escalation and expansion cohorts, the most common AEs (occurring in ≥20% of participants) were anemia, fatigue, dyspnea, nausea, and peripheral edema. The most common Grade 3 AEs were anemia and hypoxia (in ≥5% of participants). The median tₘₐₓ for MK-6482 was 1 to 2.8 hours and exposure increased with dose. The mean steady state t_{1/2} in the 120 mg QD expansion cohort (Part 1B) on Day 15 was 15.4 hours, resulting in a 1.5-fold accumulation from Day 1 to Day 15. The mean steady state Cₘₐₓ in the 120 mg QD expansion cohort (Part 1B) on Day 15 was 1.79 μg/mL (4.67 μM). The estimated CL/F was 5.22 to 14.4 L/hr. The estimated Vz/F was 106 to 266 L, which suggests extensive distribution to peripheral tissues. The CV was 32 to 59% for Cₘₐₓ and 24 to 48% for AUC after a single dose, and 27 to 56% for Cₘₐₓ and 30 to 64% for AUC at steady state. In total, 55 participants with previously treated advanced RCC have been treated in this study with MK-6482 at 120 mg QD (3 patients in the dose-escalation portion of the study and 52 participants in the dose expansion portion of the study). Best response among these 55 participants included 11 participants (20%) with PR and 32 participants (58%) with SD as assessed by RECIST v1.1.

A Phase 2, open-label efficacy and safety study in participants with VHL disease associated RCC is ongoing. As of 6 Sep. 2019, 61 participants had been enrolled at a dose of 120 mg QD. Efficacy data are not yet available. Fatigue was the most common AE of ≥Grade 3 toxicity (reported by ≥5% of participants).

In addition, 20 patients with ccRCC are being evaluated in a Phase 2 Study, and 18 healthy adult volunteers are being evaluated in a Phase 1 bioavailability study.

Based on the data from these studies, the combination of 120 mg MK6482, 400 mg of pembrolizumab, and 20 mg of lenvatinib will be evaluated in patients (1L) with advanced RCC with clear cell component (ccRCC) as a first-line therapy.

The primary objective of this study is to evaluate safety and tolerability, and the antitumor effect of the combination of MK6482, pembrolizumab and lenvatinib in patients with advanced ccRCC. This study will use ORR as the primary efficacy endpoint. ORR is defined as the percentage of participants who achieve a confirmed CR or PR per RECIST 1.1 as assessed by BICR. Responses are based on BICR using RECIST 1.1, modified to follow a maximum of target lesions and a maximum of 5 target lesions per organ. ORR is an appropriate endpoint to evaluate the antitumor activity of reference and experimental arms. Treatment effect measured by ORR can represent direct clinical benefit based on the specific disease, context of use, magnitude of the effect, number of CRs, durability of response, disease setting, location of the tumors, available therapy, and risk-benefit relationship.

The secondary objective is to evaluate DOR, PFS, OS, and CBR as secondary efficacy endpoints. “DOR” is defined as the time from the first documented evidence of CR or PR until disease progression or death due to any cause, whichever occurs first. DOR per RECIST 1.1, modified to follow a maximum of 10 target lesions and a maximum of 5 target lesions per organ, assessed by BICR will serve as an additional measure of efficacy and is a commonly accepted endpoint by both regulatory authorities and the oncology community. “PFS” is defined as the time from the date of randomization to the first documented PD per RECIST 1.1 by BICR, or death due to any cause, whichever occurs first. Images will be read by a BICR to minimize bias in the response assessments. A PFS event can reflect tumor growth and be assessed before the determination of a survival benefit. Its determination is not confounded by subsequent therapy. Treatment effect measured by PFS can be a surrogate endpoint to represent direct clinical benefit based on the specific disease, context of use, magnitude of the effect, the disease setting, location of metastatic sites, available therapy, the risk-benefit relationship, and the clinical consequences of delaying or preventing progression in key disease sites (eg, delay of new lesions in the brain or spine) or delaying administration of more toxic therapies. “OS” has been recognized as the gold standard for the demonstration of superiority of a new antineoplastic therapy in randomized clinical studies. OS is defined as the time from the date of randomization to the date of death from any cause. “CBR” is a secondary endpoint commonly used in many cancer clinical trials {059M4P} and is defined as the percentage of participants who have achieved SD of ≥6 months or CR or PR based on assessments by BICR per RECIST 1.1.

The tertiary/exploratory objectives of the study include evaluation of the correlation of tumor size change with DOR, PFS, and OS; characterization of the pharmacokinetic (PK) profiles and anti-drug antibody (ADA) formation for the investigational agents; and to identify molecular (genomic, metabolic, and/or proteomic) biomarkers that may be indicative of clinical response/resistance, safety, and/or the mechanism of action of study treatment combinations with pembrolizumab, lenvatinib and MK6482. Tumor size change will be an exploratory efficacy endpoint and is a proposed intermediate endpoint that may detect signals of early antitumor activity and is defined as the sum of target lesions in longest diameter at each post-baseline assessment and change (and % change) from baseline.

Male and female patients at least 18 years of age with advanced ccRCC who have not received prior systemic treatment for advanced disease (1L RCC) are enrolled in this study. Patients must have a histologically confirmed diagnosis of locally advanced/metastatic ccRCC (with or without sarcomatoid features), ie, Stage IV RCC per AJCC, and have received no prior systemic therapy for advanced RCC. In addition, patients must have measurable disease per RECIST 1.1 as assessed by BICR.

The reference arm of the study is described below:

| | Dose | Unit | | Route of | Regimen/ |
|---|---|---|---|---|---|
| Intervention | Formu- | Dose | Dosage | Admini- | Treatment |
| Name | lation | Strength(s) | Level(s) | stration | Period |
| Pembrolizumab | Solution | 25 | 400 mg | IV | Q6W |
| | for | mg/mL | | Infusion | |
| | Infusion | | | | |
| Lenvatinib | Capsule | 10 mg | 20 mg | Oral | QD |
| | | and 4 | | | |
| | | mg | | | |

IV = intravenous;
Q6W = every 6 weeks;
QD = once a day
NOTE:
PEMBROLIZUMAB IN THIS ARM WILL BE ADMINISTERED AS AN IV INFUSION OVER 30 MINUTES. LENVATINIB WILL BE ADMINISTERED 30 MINUTES AFTER INFUSION IS COMPLETE

### The Investigational Agents (Arm A4) for the Study are Described Below:

| | Dose | | | Route of | Regimen/ |
|---|---|---|---|---|---|
| Intervention | Formu- | Unit Dose | Dosage | Admini- | Treatment |
| Name | lation | Strengths | Levels | stration | Period |
| MK-6482 ^{d} | Tablet | 40 mg | 120 mg, | Oral | QD |
| | | | 80 mg | | |
| Pembrolizumab ^{e} | Solution | 25 | 400 mg | IV | Q6W |
| | for | mg/mL | | | |
| | Infusion | | | Infusion | |
| Lenvatinib ^{f} | Capsule | 10 mg | 20 mg, | Oral | QD |
| | | and | 14 mg, | | |
| | | 4 mg | 10 mg | | |

IV = intravenous;
Q6W = every 6 weeks;
QD = once a day.
^{d} MK-6482 will be administered 30 minutes after any infusion (if applicable) is complete.
^{e} Pembrolizumab in this arm will be administered as an IV infusion over 30 minutes.
^{f} Lenvatinib will be administered last, 30 minutes after any other agents have been administered (ie: 30 minutes after infusion(s) are complete and/or oral medication has been administered).

### Medication Dose Levels for Experimental Arm A4:

| | Dose | Dose | Dose | Dose |
|---|---|---|---|---|
| Arm A4 | Level 0 | Level-1 | Level-2 | Level-3 |
| Pembrolizumab | 400 mg | 400 mg | 400 mg | 400 mg |
| Lenvatinib | 20 mg | 14 mg | 10 mg | 10 mg |
| MK-6482 | 120 mg | 120 mg | 120 mg | 80 mg |

### Example 2

### VHL Proficient Mouse Syngeneic Pancreatic Tumor Model Combining an Anti-PD-1 Mouse Surrogate Antibody (muDX400) and a VEGF Tyrosine Kinase Inhibitor, Lenvatinib, with a HIF-2α Inhibitor, MK-6482

Here we provide preclinical data using a VHL proficient mouse syngeneic pancreatic tumor model to demonstrate the anti-tumor benefit from combining an anti-PD-1 mouse surrogate antibody (muDX400) and a VEGF tyrosine kinase inhibitor, lenvatinib, with the HIF-2α inhibitor, MK-6482.

Prior to treatment initiation, female C57BL/6J mice aged 7 weeks weighing between 18 to 21 grams were anesthetized and injected into the rear flank with 0.5×10⁶ log-phase sub-confluent KPC-2838c3 cells. Ten (10) days later when the mean tumor volume of inoculated animals reached approximately 95 mm³, mice were pair-matched into 8 treatment groups consisting of 10 mice per group. Treatment groups consisted of: 1) 0.5% methylcellulose (Vehicle)+Isotype mouse IgG1 antibody (mIgG1); 2) MK-6482+mIgG1; 3) Lenvatinib+mIgG1; 4) Vehicle+anti-mouse PD-1 IgG1 antibody; 5) MK-6482+anti-PD-1; 6) Lenvatinib+anti-PD-1; 7) MK-6482+Lenvatinib; 8) MK-6482+Lenvatinib+anti-PD-1. Vehicle and MK-6482 were orally gavage-dosed twice daily (BID) at 3 mg/kg body weight. Lenvatinib was orally dosed once daily (QD) at 10 mg/kg body weight. Isotype control, a mouse monoclonal antibody specific for adenoviral hexon of the isotype IgG1, and anti-PD-1 antibodies were dosed intraperitoneally every 5 days at 10 mg/kg body weight. Start of treatments was considered Day 0 and dosing based on schedules continued as described until Day 45. Caliper measurements of tumors and body weights were captured twice weekly. Statistical analyses were performed by one-way ANOVA with Tukey multiple comparison tests at the timepoints specified when treatment groups reached endpoints.

Triple combination treatment with MK-6482, anti-PD-1 and lenvatinib demonstrated notable anti-tumor efficacy (FIG. 2A) such that one complete response was observed where no measurable tumor remained as well as one partial tumor regression (FIG. 2B). Complete and partial tumor regressions were not observed in any other treatment groups. The mean anti-tumor response of the triple combination treatment was greater than the anti-tumor response observed with either MK-6482 monotherapy, anti-PD-1 monotherapy, or vehicle control (p<0.0001, Day 24). The triple combination group was also significantly improved over lenvatinib monotherapy (p=0.0002, Day 45). The mean anti-tumor response of the triple combination was improved over MK-6482+anti-PD-1 (p<0.0001, Day 31), but not over Lenvatinib+anti-PD-1 (p=0.247, Day 45) or Lenvatinib+MK-6482 (p=0.404, Day 45). A summary of tumor growth inhibitions (TGI) at Day 24 and observations of partial tumor regressions (PRs) or complete tumor regressions (CRs) when vehicle treated group exited the study is shown in the table below:

| Treatment | TGI (p-value) | Final PRs or CRs |
|---|---|---|
| MK-6482 | 16% (p = 0.173) | 0/10 PRs, 0/10 CRs |
| Lenvatinib | 90% (p < 0.0001) | 0/10 PRs, 0/10 CRs |
| Anti-PD-1 | 11% (p < 0.0001) | 0/10 PRs, 0/10 CRs |
| MK-6482 + anti-PD-1 | 52% (p < 0.0001) | 0/10 PRs, 0/10 CRs |
| Lenvatinib + anti-PD-1 | 89% (p < 0.0001) | 0/10 PRs, 0/10 CRs |
| Lenvatinib + MK-6482 | 92% (p < 0.0001) | 0/10 PRs, 0/10 CRs |
| Lenvatinib + MK-6482 + | 97% (p < 0.0001) | 1/10 PR, 1/10 CR |
| anti-PD-1 | | |

CRs were defined as no observable tumor whereas PRs were tumors whose volume was lower than the original tumor size when treatment began. There was no significant body weight loss or adverse events observed in any animals treated with the above therapies, indicating the treatments were well tolerated.

As shown by the study above, treatment with the combination of therapeutic agents is advantageous over treatment with each agent when administered alone.

### II. SEQUENCE LISTING

The Table below summarizes all sequences disclosed in the present specification.

### SEQ ID NOS and Corresponding Sequences

| SEQ | | |
|---|---|---|
| ID NO | Description | Sequence |
| 1 | Pembrolizumab, | RASKGVSTSGYSYLH |
| | VL-CDR1 | |
| 2 | Pembrolizumab, | LASYLES |
| | VL-CDR2 | |
| 3 | Pembrolizumab, | QHSRDLPLT |
| | VL-CDR3 | |
| 4 | Pembrolizumab, | EIVLTQSPATLSLSPGERATLSCRASKGVSTSGYSYLHWY |
| | VL | QQKPGQAPRLLIYLASYLESGVPARFSGSGSGTDFTLTIS |
| | | SLEPEDFAVYYCQHSRDLPLTFGGGTKVEIK |
| 5 | Pembrolizumab, | EIVLTQSPATLSLSPGERATLSCRASKGVSTSGYSYLHWY |
| | light chain | QQKPGQAPRLLIYLASYLESGVPARFSGSGSGTDFTLTIS |
| | | SLEPEDFAVYYCQHSRDLPLTFGGGTKVEIKRTVAAPSV |
| | | FIFPPSDEQLKSGTASVVCLLNNFYPREAKVQWKVDNAL |
| | | QSGNSQESVTEQDSKDSTYSLSSTLTLSKADYEKHKVYA |
| | | CEVTHQGLSSPVTKSFNRGEC |
| 6 | Pembrolizumab, | NYYMY |
| | VH-CDR1 | |
| 7 | Pembrolizumab, | GINPSNGGTNFNEKFKN |
| | VH-CDR2 | |
| 8 | Pembrolizumab, | RDYRFDMGFDY |
| | VH-CDR3 | |
| 9 | Pembrolizumab, | QVQLVQSGVEVKKPGASVKVSCKASGYTFTNYYMYWV |
| | VH | RQAPGQGLEWMGGINPSNGGTNFNEKFKNRVTLTTDSS |
| | | TTTAYMELKSLQFDDTAVYYCARRDYRFDMGFDYWGQ |
| | | GTTVTVSS |
| 10 | Pembrolizumab, | QVQLVQSGVEVKKPGASVKVSCKASGYTFTNYYMYWV |
| | heavy chain | RQAPGQGLEWMGGINPSNGGTNFNEKFKNRVTLTTDSS |
| | | TTTAYMELKSLQFDDTAVYYCARRDYRFDMGFDYWGQG |
| | | TTVTVSSASTKGPSVFPLAPCSRSTSESTAALGCLVKDY |
| | | FPEPVTVSWNSGALTSGVHTFPAVLQSSGLYSLSSVVTV |
| | | PSSSLGTKTYTCNVDHKPSNTKVDKRVESKYGPPCPPCP |
| | | APEFLGGPSVFLFPPKPKDTLMISRTPEVTCVVVDVSQED |
| | | PEVQFNWYVDGVEVHNAKTKPREEQFNSTYRVVSVLTV |
| | | LHQDWLNGKEYKCKVSNKGLPSSIEKTISKAKGQPREPQ |
| | | VYTLPPSQEEMTKNQVSLTCLVKGFYPSDIAVEWESNGQ |
| | | PENNYKTTPPVLDSDGSFFLYSRLTVDKSRWQEGNVFSC |
| | | SVMHEALHNHYTQKSLSLSLGK |

1. A method of treating cancer or von-Hippel Lindau disease, comprising administering to a human patient in need thereof:
(a) a PD-1 antagonist;
(b) a HIF-2α inhibitor; and
(c) lenvatinib, or a pharmaceutically acceptable salt thereof,
wherein the PD-1 antagonist is not atezolizumab.

2. The method of claim 1, wherein the cancer is selected from the group consisting of bladder cancer, breast cancer, non-small cell lung cancer, colorectal cancer, renal cell carcinoma (RCC), hepatocellular carcinoma, pancreatic cancer and melanoma.

3. The method of claim 2, wherein the cancer is RCC.

4. The method of claim 3, wherein the RCC is advanced RCC.

5. The method of claim 4, wherein the RCC is advanced RCC with clear cell component (ccRCC).

6. The method of claim 5, wherein the human patient has not received prior systemic treatment for advanced disease.

7. The method of claim 3, wherein the RCC is metastatic RCC.

8. (canceled)

9. A kit comprising:
(a) a PD-1 antagonist;
(b) a HIF-2α inhibitor; and
(c) lenvatinib, or a pharmaceutically acceptable salt thereof,
wherein the PD-1 antagonist is not atezolizumab.

10-18. (canceled)

19. The method of claim 1, wherein the PD-1 antagonist is an anti-human PD-1 monoclonal antibody or antigen binding fragment thereof.

20. (canceled)

21. The method of claim 19, wherein the anti-human PD-1 monoclonal antibody is a humanized antibody.

22. The method of claim 19, wherein the anti-human PD-1 monoclonal antibody is a human antibody.

23. The method of claim 1, wherein the HIF-2α inhibitor is belzutifan, or a pharmaceutically acceptable salt thereof.

24. (canceled)

25. The method of claim 19, wherein the anti-human PD-1 monoclonal antibody is pembrolizumab.

26. (canceled)

27. (canceled)

28. The method of claim 1, wherein:
(a) the PD-1 antagonist is pembrolizumab; and
(b) the HIF-2α inhibitor is belzutifan, or a pharmaceutically acceptable salt thereof.

29. (canceled)

30. (canceled)

31. The method of claim 28, wherein the human patient is administered 200 mg, 240 mg, or 2 mg/kg pembrolizumab, and wherein pembrolizumab is administered once every three weeks.

32. The method of claim 28, wherein the human patient is administered 400 mg pembrolizumab, and wherein pembrolizumab is administered once every six weeks.

33-34. (canceled)

35. The method of claim 31, wherein the human patient is administered from about 40 mg to about 120 mg of belzutifan, and wherein belzutifan is administered once-daily.

36. (canceled)

37. The method of claim 36, wherein the human patient is administered 120 mg of belzutifan.

38. The method of claim 31, wherein the human patient is administered 8, 10, 12, 14, 18, 20, or 24 mg lenvatinib, and wherein lenvatinib is administered once daily.

39. A method of treating RCC, comprising administering to a human patient in need thereof:
(a) 200 mg pembrolizumab;
(b) 120 mg of belzutifan, or a pharmaceutically acceptable salt thereof; and
(c) 20 mg lenvatinib.

40-43. (canceled)
